(19) United States
(12) Patent Application Publication
(10) Pub. No.: US 2022/0404355 A1
(43) Pub. Date: Dec. 22, 2022

(54) DEVICE AND METHODS FOR DIAGNOSIS OF ACTIVE TUBERCULOSIS

(71) Applicant: COLORADO STATE UNIVERSITY RESEARCH FOUNDATION, Fort Collins, CO (US)

(72) Inventors: Delphi CHATTERJEE, Fort Collins, CO (US); Charles S. HENRY, Fort Collins, CO (US)

(73) Assignee: COLORADO STATE UNIVERSITY RESEARCH FOUNDATION, Fort Collins, CO (US)

(21) Appl. No.: 17/842,210

(22) Filed: Jun. 16, 2022

Related U.S. Application Data
(60) Provisional application No. 63/211,325, filed on Jun. 16, 2021.

(51) Int. Cl. G01N 33/558; G01N 33/52; G01N 33/543; B01L 3/00

(52) U.S. Cl. CPC G01N 33/558; G01N 33/525; G01N 33/54366; B01L 3/50273; B01L 2300/069; B01L 2300/0867; B01L 2300/0887

(57) ABSTRACT

The present invention relates generally to an assay for detecting and differentiating single or multiple analytes, if present, in a fluid sample, including devices and methods of use of the same.

### RELATED APPLICATIONS

This application claims priority under 35 U.S.C. § 119(e) to U.S. Provisional Patent Application No. 63/211,325, filed Jun. 16, 2021, which is incorporated herein by reference.

### STATEMENT REGARDING FEDERAL FUNDING

This invention was made with government support under grant RO1 AI132680 awarded by National Institutes of Health. The government has certain rights in the invention.

### BACKGROUND OF THE INVENTION

Enzyme-linked immunosorbent assays (ELISA) are a popular immunodiagnostic method for clinical, environmental, and food safety testing. As a result of enzyme labeling, ELISA can detect low levels of target analytes through signal amplification. Moreover, the affinity of antigen-antibody binding typically results in high assay specificity. However, ELISA is difficult to implement in point-of-care testing (POCT) devices as it requires a laboratory setting, multiple tedious sample preparation steps, powered instrumentation, and long analysis times.

Conventional lateral flow immunoassays (LFIAs) are widely used at the POC and are based on antigen-antibody binding on a nitrocellulose membrane. LFIAs are much easier to use than ELISAs and in many cases can be performed with only a single sample loading step. The typical labeling agent for LFIAs is gold nanoparticles (AuNPs). A colored line of AuNPs appears when an immunocomplex is formed, indicating a positive result. However, AuNP LFIA sensitivity is poor in comparison to ELISA because there is no signal amplification. As a result, detection of low levels of any analyte is difficult. Enzyme-labeled LFIA platforms can improve assay sensitivity using the catalytic activity of an enzyme. Sensitivity using enzyme-labels is 50-100 times higher than AuNPs-based LFIAs. However, enzymatic LFIAs require multiple timed steps to wash and add reagents in a specified order. Typically, these extra steps complicate the assay, making the LFIA platforms difficult to be used in a POCT setting.

To overcome limitations associated with current enzyme based LFIAs, an automated ELISA integrated with an LFIA platform has been reported that allows an enzyme amplified assay to be completed in a single step. In these reports, nitrocellulose membranes were designed with delayed and non-delayed channels to sequentially rehydrate and deliver reagents spotted in different strategic locations along the nitrocellulose to a test zone. After sample loading, all reagents downstream of the non-delayed channel move together through the patterned nitrocellulose membrane to the detection zone, but the reagents dried in the delayed channel arrive after those in the non-delayed channel. Therefore, this system did not require additional washing and/or substrate addition steps during the assay. However, sequential flow in this platform relied on patterning nitrocellulose membranes, which is difficult, results in poor flow control, and is limited in the total wash and sample volume the system can process.

Accordingly, there is a need for a simple, robust, cost effective, and adaptable microfluidic testing device with high sensitivity for the target analyte. The present invention satisfies these needs.

### SUMMARY OF THE INVENTION

Among lateral flow immunoassay (LFIA) platforms, enzyme-based LFIAs provide signal amplification to improve sensitivity. However, most enzyme-based LFIAs require multiple timed steps, complicating their utility in point-of-care testing (POCT). The present microfluidic interface for LFIAs that automates sample, buffer, and reagent addition, greatly simplifying operation while achieving the high analytical stringency associated with more complex assays. The microfluidic interface also maintains the low cost and small footprint of standard LFIAs. The platform is fabricated from a combination of polyester film, double-sided adhesive tape, and nitrocellulose, and fits in the palm of your hand. All reagents are dried on the nitrocellulose to facilitate sequential reagent delivery, and the sample is used as the wash buffer to minimize steps. After the sample addition, a user simply waits 15 min for a colorimetric result.

The disclosed microfluidic interface combined with a nitrocellulose membrane facilitates an enzyme amplified LFIAs that uses a simple fabrication. The assay is performed on a nitrocellulose membrane which connects to an absorbent pad at the downstream end. Reagents are automatically delivered to the detection zone on the nitrocellulose in a controlled order via flow from two outlets of the microfluidic interface. First, it was confirmed that the new microfluidic interface automates the immunoreagent delivery. Next, lipoarabinomannan (LAM), a lipopolysaccharide antigen, which is a urinary biomarker for tuberculosis (TB) was detected using the developed device. Detecting LAM in urine has been confirmed as a promising TB diagnostic method in different clinical populations as well as patients with advanced immunodeficiency and low CD4 cell counts. Also, the POC platform of LAM detection enables immediate TB treatment for high-risk patients. In one embodiment, the assay used horseradish peroxidase (HRP) labeled antibodies and 3,3′-diaminobenzidine tetrahydrochloride (DAB) as the colorimetric substrate. To achieve the best assay performance, the parameters for each step of the assay were optimized. Next, several different concentrations of LAM in PBS were detected using the fully optimized system. Finally, the reliability and feasibility of the microfluidic interface was confirmed by determining LAM spiked in urine samples.

Accordingly, this disclosure provides for a microfluidic device and methods of use of said device for detecting an analyte in a sample. In one embodiment, the microfluidic device comprises a testing zone comprising a nitrocellulose membrane comprising a proximal end, a distal end, and a center region, wherein the testing zone comprises an antibody zone disposed between the distal end and the center region of the testing zone, wherein the antibody zone comprises, in order from the center region to the distal end: a detection zone comprising mobilizable detection antibodies conjugated to a labeling component and spot-dried to a surface of the detection zone; a capture zone comprising one or more capture antibodies that are spot-dried and immobilized on a surface of the capture zone; and a control zone comprising one or more anti-mobilizable detection antibodies that are spot-dried and immobilized on a surface of the control zone; a substrate component and hydrogen peroxide separately spot-dried on a surface of the testing zone between the proximal end of the testing zone and the center region of the testing zone; a sample inlet for receiving a sample comprising: a first sample outlet intersecting with, and in fluid communication with, the center region of the testing zone; a second sample outlet fluidly connected to a first flow channel, wherein the first flow channel is in fluid communication with the proximal end of the testing zone; and an absorbent pad in fluid communication with the distal end of the testing zone; wherein the first flow channel has a greater length than the length of the first sample outlet.

One embodiment of a method of using the microfluidic device to detect a target analyte in a test sample comprises: a) contacting the microfluidic device with the test sample comprising one or more target analytes and one or more buffer components, wherein the test sample is received in the sample inlet, wherein a first fraction of the test sample migrates by capillary action through the first sample outlet to contact the center region of the testing zone, wherein the first fraction of the test sample flows toward both the proximal end of the testing zone and the distal end of the testing zone, wherein the first fraction of the testing sample rehydrates, spreads, and mixes the desorbed mobilizable detection antibody conjugated to a labeling component over the antibody zone; b) binding the desorbed mobilizable detection antibody to the one or more target analytes to form an analyte-antibody complex, wherein the analyte-antibody complex then binds to the immobilized capture antibody, and the immobilized anti-detection antibody specifically binds to desorbed and unbound mobilizable detection antibody; c) migrating, by capillary action, the second fraction of the test sample through the flow channel towards the distal end of the testing zone such that the second fraction rehydrates, spreads, and mixes the substrate component and the hydrogen peroxide over the testing zone; d) detecting a signal from the analyte-antibody complex bound to the immobilized capture antibody, the desorbed and unbound mobilizable detection antibody bound attached to the immobilized anti-detection antibody, or a combination thereof, wherein a detectable signal from both the analyte-antibody complex bound to the immobilized capture antibody and the desorbed and unbound detection antibody attached to the immobilized anti-detection antibody indicates the presence of the target analyte in the test sample.

In another embodiment, a method of determining the presence or absence of a target analyte in a test sample comprises the steps of contacting a device as described herein with a sample; forming a complex comprising the target analyte specifically bound to the mobilizable detection antibody; and measuring a detectable signal produced by: a) both the complex specifically bound to the immobilized capture antibody and the mobilizable detection antibody not attached to the complex that specifically binds to the immobilized anti-detection antibody; or b) the mobilizable detection antibody not attached to the complex that specifically binds to the immobilized anti-detection antibody; thereby determining the presence of the target analyte in the test sample if the detectable signal is produced as recited in part a) and the absence of the target analyte in the test sample if the detectable signal is produced as recited in part b).

These and other features and advantages of this invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. Schematics of the microfluidic interface. (a) Top view, (b) exploded view, and (c) reagent positions on the nitrocellulose membrane. (d) The actual image of the device including flow channel, nitrocellulose membrane, and absorbent pad.

FIG. 2. Illustration of the assay's detection step. (a) adding the sample solution, (b) solution flowing direction and formation of immunocomplex at detection zone, (c) substrate passing over the capture strip and the results with and without LAM in the system.

FIG. 3. (a) Schematic of sample and dye flow on a nitrocellulose membrane. (b) Actual images of dye flow with different injection volumes.

FIG. 4. Optimization of effecting parameters: (a) Capture Ab concentration at test line, (b) the amount of detection Ab, (c) Ratio of substrate concentration (DAB (mg/mL)/H₂O₂(%)), and (d) assay time for LAM detection.

FIG. 5. (a) Image results of dose response curve using the proposed device and (b) dose response curve between LAM concentration in 10 mM PBS, pH 7.4 VS. ΔI % in gray scale for LAM analysis.

FIG. 6. Effect of (a) the amount of polyvinylpylolidone (% PVP) by fixing the amount of Triton X-100 at 2% and (b) the amount of Triton X-100 (%) by fixing the amount of PVP at 5% and LAM concentration at 1 μg mL⁻¹ on assay sensitivity.

FIG. 7. Effect of H₂O₂ form on signal intensity (a) dry and (b) fresh.

### DETAILED DESCRIPTION

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001 or Singleton, et al., Dictionary of Microbiology and Molecular Biology, 2d ed., John Wiley and Sons, New York (1994), and Hale & Markham, The Harper Collins Dictionary of Biology. Harper Perennial, N.Y. (1991). General laboratory techniques (DNA extraction, RNA extraction, cloning, cell culturing. etc.) are known in the art and described, for example, in Molecular Cloning: A Laboratory Manual, J. Sambrook et al., 4th edition, Cold Spring Harbor Laboratory Press, 2012.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five substituents on the ring.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number 1” to “number 2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number 10”, it implies a continuous range that includes whole numbers and fractional numbers less than number 10, as discussed above. Similarly, if the variable disclosed is a number greater than “number 10”, it implies a continuous range that includes whole numbers and fractional numbers greater than number 10. These ranges can be modified by the term “about”, whose meaning has been described above.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

Alternatively, the terms “effective amount” or “therapeutically effective amount,” as used herein, refer to a sufficient amount of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the compositions, the type or extent of supplemental therapy used, ongoing disease process and type of treatment desired (e.g., aggressive vs. conventional treatment).

As used herein, “subject” or “patient” means an individual having symptoms of, or at risk for, a disease or other malignancy. A patient may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods provided herein, the mammal is a human.

As used herein, the terms “providing”, “administering,” “introducing,” are used interchangeably herein and refer to the placement of a compound of the disclosure into a subject by a method or route that results in at least partial localization of the compound to a desired site. The compound can be administered by any appropriate route that results in delivery to a desired location in the subject.

The compounds and compositions described herein may be administered with additional compositions to prolong stability and activity of the compositions, or in combination with other therapeutic drugs.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation, or limitations not specifically disclosed herein.

“Biomarker” means a molecule or molecules associated with a physiological condition of health or pathology in a vertebrate. Biomarkers may include not only the proteome, genome and metabolome of the vertebrate host, but also the proteome, genome and metabolome of normal flora or pathogenic infectious agents of the vertebrate body, including bacterial, protozoan, and viral pathogens. Preferred biomarkers include antigens and antibodies.

A “biological sample” means representative biosamples including, but not limited to, blood, serum, plasma, buffy coat, wound exudates, pus, lung and other respiratory aspirates, nasal aspirates, bronchial lavage fluids, saliva, sputum, medial and inner ear aspirates, cyst aspirates, cerebral spinal fluid, feces, urine, tears, mammary secretions, ovarian contents, ascites fluid, mucous, stomach fluid, gastrointestinal contents, urethral discharge, synovial fluid, peritoneal fluid, vaginal fluid or discharge, amniotic fluid, semen or the like. Assay from swabs or lavages representative of mucosal secretions and epithelia are also anticipated, for example mucosal swabs of the throat, tonsils, gingival, nasal passages, vagina, urethra, anus, and eyes, as are homogenates, lysates and digests of tissue specimens of all sorts. Besides physiological fluids, samples of water, food products, air filtrates, and so forth may also be test specimens.

“Immunosorbent” is understood in the context of an analyte-sorbent complex or antibody-sorbent complex for use in immunoassays as a solid-phase capture surface. Preferred sorbent materials have relatively high surface areas and are wettable under assay conditions. Sorbent materials that have been successfully “decorated” with capture agent or antibody include agarose in bead form, such as Sephadex, other carbohydrates such as dextran, cellulose and nitrocellulose, plastics such as polystyrene, polycarbonate, polypropylene and polyamide, inorganic substrates such as glass, silica gel and aluminum oxide, and high molecular weight cross-linked proteins. Plastics are optionally plasma-treated to improve binding and may be masked during plasma treatment to localize binding sites in the test field. Immunosorbent materials may be fabricated and used in the form of particles, beads, mats, sponges, filters, fibers, plates, and the like.

“Microfluidic channel”, also termed “flow channel”, means a fluid channel having variable length, but cross-sectional area often less than 500 μm, in some cases twice that, as when the sample contains particles or a bead reagent is used. Microfluidic fluid flow behavior in a microfluidic channel is highly non-ideal and laminar, as in Poiseuille flow, and may be more dependent on wall wetting properties and diameter than on pressure drop. Hybrid microscale and microfluidic devices are encompassed here. Microfluidic channel surfaces may be passivated if desired.

Masking is commonly used to define boundaries within which the capture molecule will be fixed to the plastic surface. Masking to mark out a test site aids in visual recognition of a positive assay and also in machine-aided image analysis of automated test results. Plastic surfaces may be passivated outside the defined boundaries of the mask, or in negative masking techniques, the plastic surface will be activated, such as by low pressure gas plasma treatment, where unmasked.

The term “specific binding” or “specific interaction” is the specific recognition of one of two different binding entities for the other compared to substantially less recognition of other molecules. Generally, the molecules have areas on their surfaces or in cavities giving rise to specific recognition between the two molecules. Exemplary of specific binding are antibody-antigen interactions, enzyme-substrate interactions, polynucleotide hybridization interactions, and so forth.

“Antigen” means any compound capable of binding to an antibody, or against which antibodies can be raised.

“Antibody” refers to a polypeptide substantially encoded by an immunoglobulin gene or immunoglobulin genes, or fragments thereof. The recognized immunoglobulin genes include the kappa, lambda, alpha, gamma, delta, epsilon, and mu constant regions, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. Typically, an antibody is an immunoglobulin having an area on its surface or in a cavity that specifically binds to and is thereby defined as complementary with a particular spatial and polar organization of another molecule. The antibody can be polyclonal or monoclonal. Antibodies may include a complete immunoglobulin or fragments thereof. Fragments thereof may include Fab, Fv and F(ab′)2, Fab′, and the like. Antibodies may also include chimeric antibodies or fragment thereof made by recombinant methods.

“Analyte” refers to the compound or composition to be detected or measured and which has at least one epitope or binding site. The analyte can be any substance for which there exists a naturally occurring analyte specific binding member or for which an analyte-specific binding member can be prepared. e.g., carbohydrate and lectin, hormone and receptor, complementary nucleic acids, and the like. Further, possible analytes include virtually any compound, composition, aggregation, or other substance which may be immunologically detected. That is, the analyte, or portion thereof, will be antigenic or haptenic having at least one determinant site, or will be a member of a naturally occurring binding pair. Analytes include, but are not limited to, toxins, organic compounds, proteins, peptides, microorganisms, bacteria, viruses, amino acids, nucleic acids, carbohydrates, hormones, steroids, vitamins, drugs (including those administered for therapeutic purposes as well as those administered for illicit purposes), pollutants, pesticides, and metabolites of or antibodies to any of the above substances. The term analyte also includes any antigenic substances, haptens, antibodies, macromolecules, and combinations thereof. A non-exhaustive list of exemplary analytes is set forth in U.S. Pat. Nos. 4,366,241, 4,299,916; 4,275,149; and 4,806,311, all incorporated herein by reference.

“Label” or “labeling component” refers to any substance which is capable of producing a signal that is detectable by visual or instrumental means. Various labels suitable for use in the present invention include labels which produce signals through either chemical or physical means. Such labels can include enzymes and substrates, chromogens, catalysts, fluorescent compounds, chemiluminescent compounds, and radioactive labels. Other suitable labels include particulate labels such as colloidal metallic particles such as gold, colloidal non-metallic particles such as selenium or tellurium, dyed or colored particles such as a dyed plastic or a stained microorganism, organic polymer latex particles and liposomes, colored beads, polymer microcapsules, sacs, erythrocytes, erythrocyte ghosts, or other vesicles containing directly visible substances, and the like. Typically, a visually detectable label is used as the label component of the label reagent, thereby providing for the direct visual or instrumental readout of the presence or amount of the analyte in the test sample without the need for additional labeling at the detection sites.

The selection of a particular labeling component is not critical to the present invention, but the label will be capable of generating a detectable signal either by itself, or be instrumentally detectable, or be detectable in conjunction with one or more additional labeling components, such as an enzyme/substrate labeling system. A variety of different label reagents can be formed by varying either the label or the specific binding member component of the label reagent; it will be appreciated by one skilled in the art that the choice involves consideration of the analyte to be detected and the desired means of detection. As discussed below, a label may also be incorporated used in a control system for the assay.

For example, one or more labeling components can be reacted with the label to generate a detectable signal. If the label is an enzyme, then amplification of the detectable signal is obtained by reacting the enzyme with one or more substrates or additional enzymes and substrates to produce a detectable reaction product.

In an alternative signal producing labeling system, the label can be a fluorescent compound where no enzymatic manipulation of the label is required to produce the detectable signal. Fluorescent molecules include, for example, fluorescein, phycobiliprotein, rhodamine and their derivatives and analogs are suitable for use as labels in such a system.

The use of dyes for staining biological materials, such as proteins, carbohydrates, nucleic acids, and whole organisms is documented in the literature. It is known that certain dyes stain particular materials preferentially based on compatible chemistries of dye and ligand. For example, Coomassie Blue and Methylene Blue for proteins, periodic acid-Schiff s reagent for carbohydrates, Crystal Violet, Safranin O, and Trypan Blue for whole cell stains, ethidium bromide and Acridine Orange for nucleic acid staining, and fluorescent stains such as rhodamine and Calcofluor White for detection by fluorescent microscopy. Further examples of labels can be found in, at least, U.S. Pat. Nos. 4,695,554; 4,863,875; 4,373,932; and 4,366,241, all incorporated herein by reference.

“Observable signal” as used herein refers to a signal produced in the claimed devices and methods that is detectable by visual inspection. Without limitation, the type of signal produced depends on the label reagents and marks used (described herein). Generally, observable signals indicating the presence or absence of an analyte in a sample may be evident of their own accord, e.g., plus or minus signs or particularly shaped symbols, or may be evident through the comparison with a panel such as a color indicator panel.

As used herein, “Triton X-100” (C₁₄H₂₂O(C₂H₄O)ₙ) refers to a non-ionic surfactant having a hydrophilic polyethylene oxide chain (on average it has 9.5 ethylene oxide units) and an aromatic hydrocarbon lipophilic or hydrophobic group. The hydrocarbon group is a 4-(1,1,3,3-tetramethylbutyl)-phenyl group.

### Embodiments of the Invention

This disclosure relates to microfluidic devices and methods for using and making the same. The microfluidic devices described herein may utilize microfluidic channels, inlets, valves, pumps, liquid barriers, and other elements arranged in various configurations to manipulate the flow of a liquid sample in order to qualitatively and/or quantitatively analyze the liquid sample for the presence of one or more target analytes of interest.

Generally, microfluidic devices may be constructed by a lamination process from layers of clear plastic such as polyethylene terephthalate (PET), polystyrene, polycarbonates, polyacrylates, or polyesters in general, joined by interposed layers of adhesive. Microchannels or flow channels, voids, and holes, are first machined from the plastic layers and adhesive before assembly, so that a microfluidic network is formed. Alternatively, the device may be constructed by injection molding of a cover and base layer, optionally with interposed plastic layers of increasing complexity, the layers held together with adhesive or fused under pressure with heat or solvent.

Other microfluidic devices may be fabricated from various materials using techniques such as laser stenciling, embossing, stamping, injection molding, masking, etching, and three-dimensional soft lithography. Laminated microfluidic devices are further fabricated with adhesive interlayers or by thermal adhesive-less bonding techniques, such by pressure treatment of oriented polypropylene. Fabrication of injection molded microfluidic devices may include sonic welding or UV-curing glues for assembly of parts.

Other microfluidic devices also may include a backing is typically made of water-insoluble, non-porous and rigid material and has a length and width equal to or greater than the layers situated thereon. In preparation of the backing, various natural and synthetic organic and inorganic materials can be used, provided that the backing prepared from the material should not hinder capillary actions of the absorption material, nor non-specifically bind to an analyte, nor interfere with the reaction of the analyte with a detector. Representative examples of polymers usable in the present invention include, but are not limited to, polyethylene, polyester, polypropylene, poly(4-methylbutene), polystyrene, polymethacrylate, poly(ethylene terephthalate), nylon, poly(vinyl butyrate), glass, ceramic, metal, and the like.

In some embodiments, the microfluidic devices also are formed of, or may include additional material, to permit a fluid sample to rapidly move via capillary action to reach the testing zone. Typically, this chromatography material refers to a porous material having a pore diameter of about 0.1μ to about 1.0μ, and through which an aqueous medium can readily move via capillary action. Such material generally may be hydrophilic or hydrophobic, including for example, inorganic powders such as silica, magnesium sulfate, and alumina; natural polymeric materials, particularly cellulosic materials and materials derived from cellulose, such as fiber containing papers, e.g., filter paper, chromatographic paper, etc.; synthetic or modified naturally occurring polymers, such as nitrocellulose, cellulose acetate, poly(vinyl chloride), polyacrylamide, cross-linked dextran, agarose, polyacrylate, etc.; either used by themselves or in conjunction with other materials. Also, ceramics may be used. The chromatography medium can be bound to the backing. Alternatively, the chromatography may be the backing per se. The chromatography medium may be multifunctional or be modified to be multifunctional to covalently bind to a means for detecting an analyte or another component such as an enzyme or a substrate component as described in more detail below.

In one embodiment, the microfluidic device is of laminated construction comprises one or more layers of hydrophilic polyester film and one or more layers of double-sided adhesive film wherein the one or more layers of double-sided adhesive film are disposed between layers of the hydrophilic polyester film.

As noted above, microfluidic systems may require some type of external fluidic driver to function, such as piezoelectric pumps, micro-syringe pumps, electroosmotic pumps, and the like. However, in U.S. Pat. No. 6,743,399, which patent is hereby incorporated by reference in its entirety, microfluidic systems are described which are completely driven by inherently available internal forces such as gravity, hydrostatic pressure, capillary force, absorption by porous material, or chemically induced pressures or vacuums.

In some embodiments, the microfluidic device comprises a nitrocellulose membrane. Preferably, the nitrocellulose membrane comprises a testing zone. The length of the nitrocellulose membrane may be about 5 mm to about 50mm in length, about 10 mm to about 40 mm in length, of about 15 mm to about 35 mm in length such that the required number of reagents (e.g., water-soluble polymer, surfactant), labeling component (e.g., an antibody), and substrate components (e.g., a colorimetric agent) may be spot-dried to the surface of the nitrocellulose membrane without interacting or unintendedly mixing with one another. Preferably, the length of the nitrocellulose membrane is about 15 mm to about 35 mm in length, or about 30 mm in length.

In some embodiments, the nitrocellulose membrane may include oner more changes in width of along the length of the nitrocellulose membrane. In one embodiment, the nitrocellulose membrane tapers from a first width comprising the detection zone to a second width comprising the capture zone and the control zone. For example, the nitrocellulose membrane may taper from 10 mm to about 9 mm, about 10 mm to about 8 mm, 10 mm to about 7 mm, about 10 mm to about 6 mm, 10 mm to about 5 mm, about 10 mm to about 4 mm, 10 mm to about 3 mm, or about 10 mm to about 2 mm. In another embodiment, the nitrocellulose membrane may taper from about 9 mm to about 8 mm, about 9 mm to about 7 mm, about 9 mm to about 6 mm, about 9 mm to about 5 mm, about 9 mm to about 74 mm, about 9 mm to about 3 mm, or about 9 mm to about 2 mm. In another embodiment, the nitrocellulose membrane may taper from about 8 mm to about 7 mm, about 8 mm to about 6 mm, about 8 mm to about 5 mm, about 8 mm to about 4 mm, about 8 mm to about 3 mm, or about 8 mm to about 2 mm. In another embodiment, the nitrocellulose membrane may taper from about 7 mm to about 6 mm, about 7 mm to about 5 mm, about 7 mm to about 4 mm, about 7 mm to about 3 mm, about 7 mm to about 2 mm. In another embodiment, the nitrocellulose membrane may taper from about 6 mm to about 5 mm, about 6 mm to about 4 mm, about 6 mm to about 3 mm, or about 6 mm to about 2 mm. In another embodiment, the nitrocellulose membrane may taper from about 5 mm to about 4 mm, about 5 mm to about 3 mm, or about 5 mm to about 2 mm. In another embodiment, the nitrocellulose membrane may taper from about 4 mm to about 3 mm, or about 4 mm to about 2 mm. In some embodiments, the testing zone may have multiple tapered sections. In other embodiments, the testing zone may have one or more tapering sections and in between the one or more tapered sections, the width of the testing zone increases back to the original width or greater than the original width. In one embodiment, the nitrocellulose membrane tapers from a first width of about 5mm to about 3 mm comprising the detection zone to a second width of about 4 mm to about 2 mm comprising the capture zone and the control zone. In one embodiment, the nitrocellulose membrane tapers from a first width of about 3 mm comprising the detection zone to a second width of about 2 mm comprising the capture zone and the control zone.

In some embodiments, the microfluidic device comprises an absorbent material known as a “absorbent pad” or “waste pack”. The absorbent pad may be constructed of any absorbent material such as filter paper or Whatman paper. The absorption pad is a means for physically absorbing the sample which has chromatographically moved through the chromatography medium via capillary action and for removing unreacted substances. Thus, the absorption pad is located at the end of the testing zone to control and promote movement of samples and reagents and acts as a pump and container for accommodating them. The speeds of samples and reagents may vary depending on the quality and size of the absorption pad. Commonly used absorption pads are formed of water-absorbing material such as cellulose filter paper, non-woven fabric, cloth, cellulose acetate, absorbent foams, absorbent sponges, superabsorbent polymers, or absorbent gelling materials. The absorbent pad can be used to migrate or propel fluid flow by capillary wetting in place of, or in concert with, microfluidic pumps.

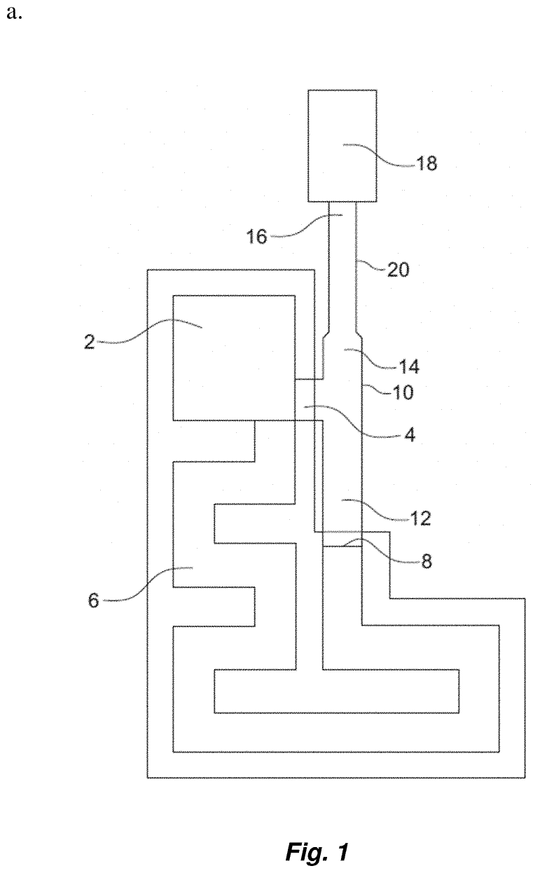

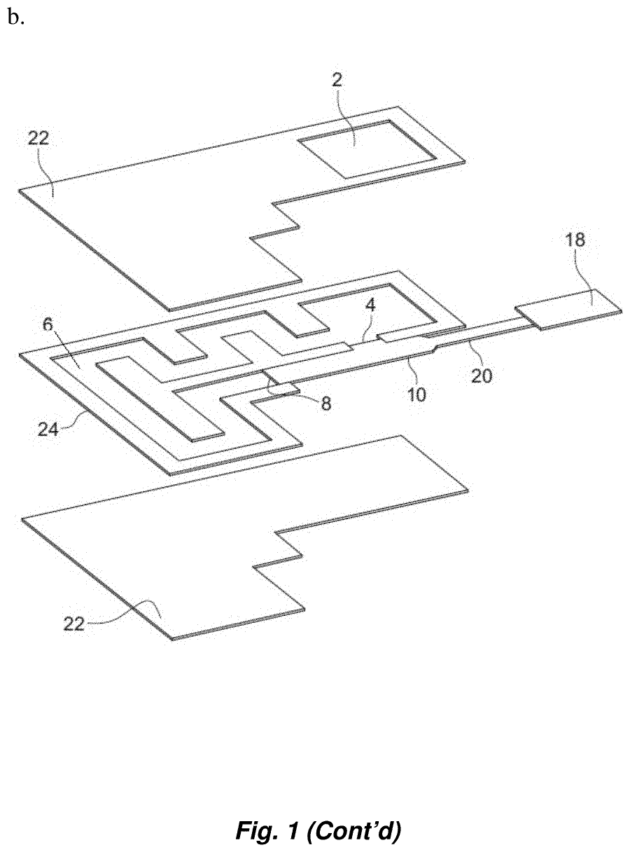

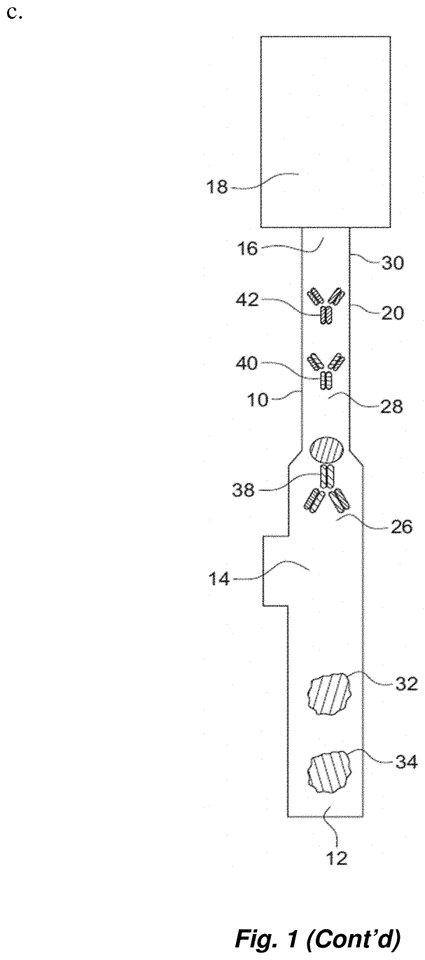

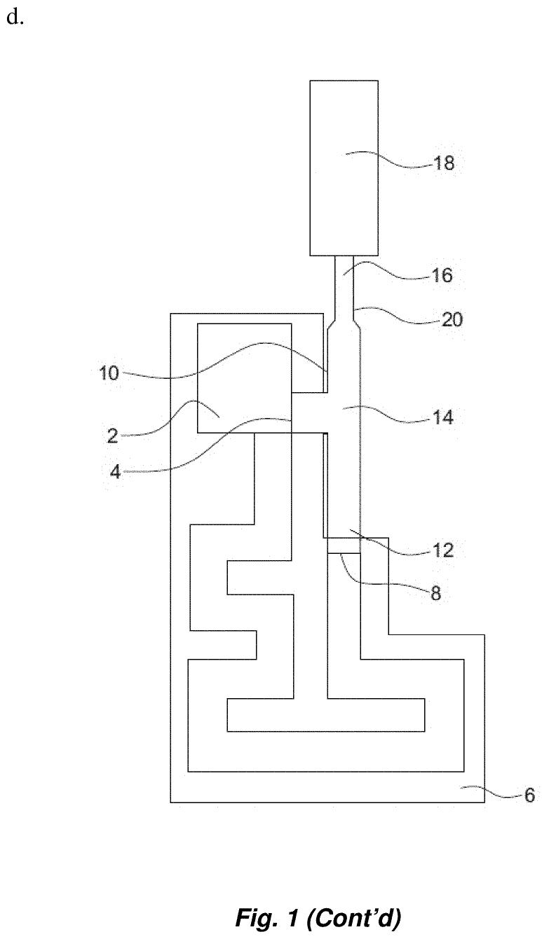

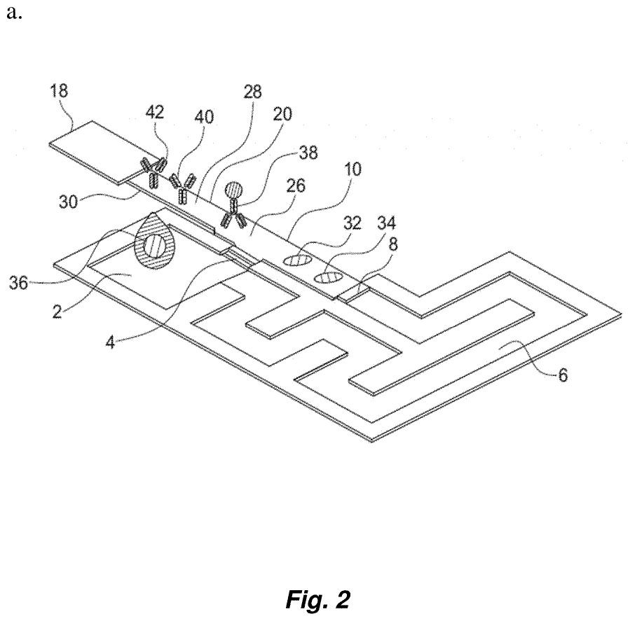

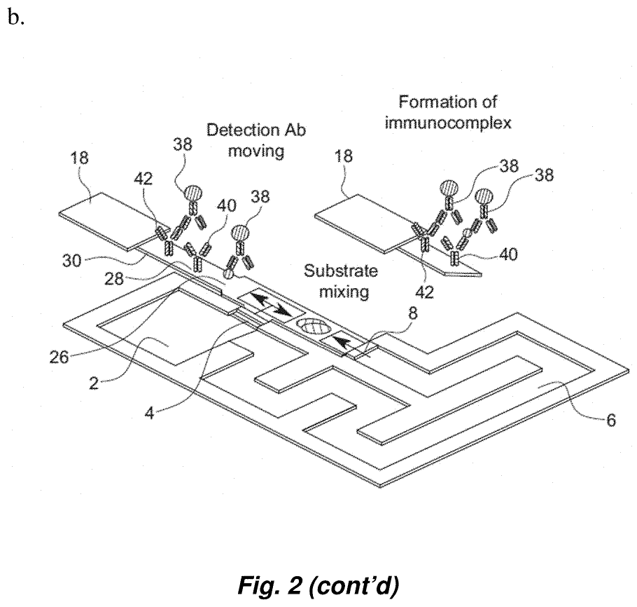

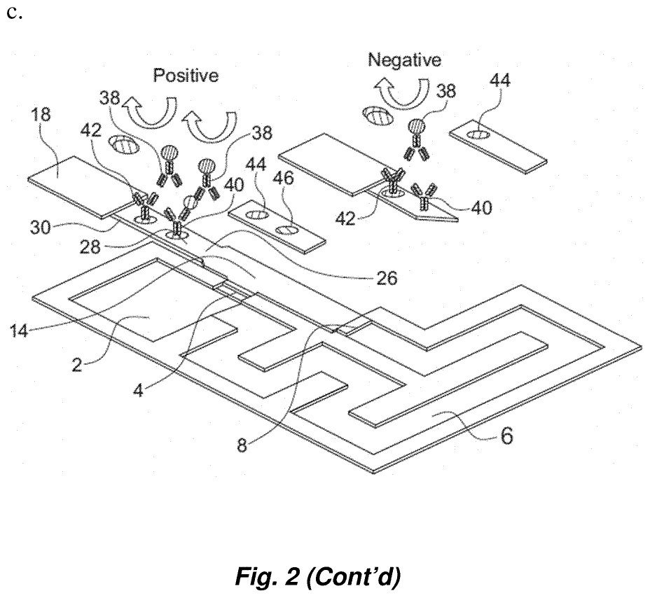

In some embodiments, the nitrocellulose membrane comprises one or more testing zones for detecting a target analyte. An exemplary testing zone is disclosed in FIG. 1 and FIG. 2, and is discussed in more detail below. Preferably, certain testing zones of the disclosure comprise an antibody zone that itself comprises one or more of: a detection zone, a capture zone, and a control zone, each of which may have one or more means of detection disposed on the surface of the antibody zone, either spot-dried and immobilized (e.g., covalently attached to the surface) to the surface or spot-dried and mobilizable (that is to say, a mobilizable means of detection may be rehydrated by the test sample and desorbed from the surface of the antibody zone to spread with the test sample as it migrates across the surface of the antibody zone). The antibody zone in the testing zone permits the use of affinity binding assays to detect the presence or absence of a target analyte. In some embodiments, the testing zone comprises at least one detection zone, at least one capture zone, and at least one control zone.

Various modes of affinity binding assays that may be used with the device, such as immunoaffinity binding assays, include, for example, immunohistochemistry methods, solid phase Enzyme-linked immunosorbent assay (ELISA), and Radioimmunoassays (RIA) as well as modifications thereof. Such modifications thereof include, for example, capture assays and sandwich assays as well as the use of either mode in combination with a competition assay format. The choice of which mode or format of immunoaffinity binding assay to use will depend on the intent of the user. Such methods can be found described in common laboratory manuals such as Harlow and Lane, Using Antibodies: A Laboratory Manual, Cold Spring Harbor Laboratory Press, New York (1999).

In some embodiments, the detection zone of the antibody zone comprises one or more mobilizable detection antibodies or other means of detection that specifically bind to the target analyte. Further, the mobilizable detection antibody is conjugated to one or more labeling components to facilitate generating a detectable signal when the target analyte is present in the testing sample.

In some embodiments, the labeling component is an enzyme such as an oxidase, peroxidase, phosphatase, diaphorase, galactosidase, lytic enzyme, or oxidoreductase. The enzyme will usually be covalently attached to the specific binding substance (e.g., a detection antibody), but indirect linkage such as through a biotin-avidin binding or other cognate members of specific binding pairs may also find use. When the specific binding substance is a polypeptide or protein, such as an antibody, the enzyme may be covalently bound through a variety of moieties, including disulfide, hydroxyphenyl, amino, carboxyl, indole, or other functional groups, employing conventional conjugation chemistry as described in the scientific and patent literature. Binding should be affected in such a way that active site(s) on the means of detection are not blocked and remain available for binding to the desired target analyte. In the case of antibodies, binding will preferably be affected so that the complementary determining regions remain available for binding to the target analyte. Specific techniques for derivatizing antibodies binding to enzymes are described in Tijssen, “Practice and Theory of Enzyme Immunoassays” in Laboratory Techniques in Biochemistry and Molecular Biology, vol. 15, Burdon and van Knippenberg, eds. 1985, Elsevier, Amsterdam, the disclosure which is incorporated herein by reference.

In some embodiments, the means of detection is a mobilizable detection antibody conjugated to a labeling component, wherein the labeling component is an enzyme comprising a peroxidase enzyme or a phosphatase enzyme. In some embodiments, an antibody (e.g., a detection antibody) is coupled to labeling component such as horseradish peroxidase (HRP) and alkaline phosphatase (ALP or AP).

In other embodiments, the labeling component is selected from the group consisting of a chemiluminescent agent, a particulate label, a colorimetric agent, an energy transfer agent, an enzyme, a fluorescent agent, and a radioisotope.

The antibody zone may further comprise a capture zone that comprises one or more capturing antibody immobilized in the capture zone through the physical adsorption or covalent binding to the nitrocellulose membrane. The capturing species is immobilized onto the analyte capture zone to capture any mobilizable detection antibody specifically bound to the target analyte that passes through the capture zone.

Alternatively, the capturing antibody can be one that directly binds to the analyte of interest, such as an analyte specific antibody. One example of capturing species of this type is one used in a sandwich-type ELISA, in which an antibody for the target analyte specifically binds to the analyte capture zone and a detecting species that also binds to the analyte of interest is present to enable detection of the presence of the analyte. In this example, the capture antibody binds to a different epitope on the analyte of interest than the detecting species (or antibody). Preferably, the capturing species can bind to a detecting species that has complexed with the analyte of interest. Another example immunoassay of this type is a biotin-streptavidin binding assay wherein the streptavidin (capturing species) is immobilized on the porous membrane at the analyte capture zone, and the biotin (detecting species) is conjugated to an antibody which binds the analyte of interest. In either case, the presence of the detecting species at the analyte capture zone may indicate the presence of the analyte of interest in the sample.

The antibody zone also comprises a control zone comprising one or more detection means immobilized on the surface of the nitrocellulose membrane. Preferably, the detection means is an antibody. The control antibody specifically binds to excess detection antibody that did not specifically bind to the target analyte either because all the target analyte is bound by the detection antibody leaving an excess of unbound detection antibody or the excess detection antibody is due the absence of the target analyte in the test sample. Accordingly, the control antibody comprises one or more anti-detection antibody. As a person of ordinary skill in the art will appreciate, capture of the detection antibody conjugated to a labeling component by either of the capture antibody or the control antibody will produce a detectable signal in the presence of a suitable substrate component if needed. In some embodiments, the substrate component is a colorimetric agent. In some embodiments, the substrate component is 3,3′-diaminobenzidine tetrahydrochloride (DAB) or similar compound that permits signal detection when the signal component is a peroxidase enzyme (e.g., horse radish peroxidase) or BCIP/NBT (5-Bromo-4-chloro-3-indolyl phosphate along with nitro blue tetrazolium), pNPP (para-Nitrophenylphosphate), and Fast Red when the signal component is a phosphatase enzyme (e.g., alkaline phosphatase).

In some embodiments, each of the mobilizable detection antibodies further comprise a mixture of a water-soluble polymer and a surfactant that is spot-dried to a certain surface as needed. Preferably, the mixture comprises about 1% v/v to about 10% v/v of the water-soluble polymer, about 2% v/v to about 9% v/v of the water-soluble polymer, about 3% v/v to about 8% v/v of the water-soluble polymer, or about 4% v/v to about 6% v/v of the water-soluble polymer. Preferably, the mixture comprises about 1% v/v to about 10% v/v of the surfactant, about 2% v/v to about 9% v/v of the surfactant, about 3% v/v to about 8% v/v of the surfactant, or about 4% v/v to about 6% v/v of the surfactant. In some embodiments, the mixture comprises about 5% v/v of the water-soluble polymer about 5% v/v of the surfactant. In other embodiments, the mobilizable detection antibody does not contain a surfactant or a water-soluble polymer.

In some embodiments, the surfactant may comprise one or more non-ionic detergents, that is, a detergent that includes molecules with head groups that are uncharged. Non-ionic detergents may comprise polyoxyethylene (and related detergents), and glycosidic compounds (e.g., alkyl glycosides). Exemplary alkyl glucosides include octyl β-glucoside, n-dodecyl-β-D-maltoside, beta-decyl-maltoside, and Digitonin. Examples of polyoxyethylene detergents include polysorbates (e.g., polysorbate 20, Polysorbate 40, polysorbate 60, polysorbate 80 (also known as TWEEN-20, TWEEN-40, TWEEN-60, and TWEEN-80, respectively), TRITON-X series (e.g., TRITON X-100), TERGITOL series of detergents (e.g., NP-40), the BRIJ series of detergents (e.g., BRIJ-35, BRIJ-58, BRIJ-L23, BRIJ-L4, BRIJ-O10), and PLURONIC F68.

In some embodiments, the water-soluble polymer of the mixture is polyvinylpyrrolidone and the surfactant of the mixture is Triton X-100. Preferably, the mixture comprises about 1% v/v to about 10% v/v of the polyvinylpyrrolidone, about 2% v/v to about 9% v/v of the polyvinylpyrrolidone, about 3% v/v to about 8% v/v of the polyvinylpyrrolidone, or about 4% v/v to about 6% v/v of the polyvinylpyrrolidone. Preferably, the mixture also comprises about 1% v/v to about 10% v/v of the Triton X-100, about 2% v/v to about 9% v/v of the Triton X-100, about 3% v/v to about 8% v/v of the Triton X-100, or about 4% v/v to about 6% v/v of the Triton X-100. In some embodiments, the mixture comprises about 5% v/v of the polyvinylpyrrolidone and about 5% v/v of the Triton X-100. Advantageously, the water-soluble polymer and surfactant mixture dried on a surface of the microfluidic device serves as a mobilizable detection antibody dilution buffer to minimize permanent adherence to the surface and non-specific binding on a non-target analyte.

As one of ordinary skill in the art will appreciate, any the foregoing reagents and antibodies may be printed onto microfluidic device (e.g., the testing zone or antibody zone) by methods such as ink jet printing, micro drop printing, and transfer printing. In other embodiments, the reagents and antibodies (or other means detection) maybe micro-pipetted spot-dried onto a surface.

In some embodiments, a microfluidic device may comprise proteinase K (ProK) disposed on a surface of one or more of the sample inlet, the first sample outlet, the second sample outlet, the first flow channel (or any flow channel present in the device), or a combination thereof. ProK is a broad serine protease and cleaves proteins at the carboxyl side of the aromatic and hydrophobic amino acids. The enzyme shows maximum activity in the pH range of 7.0-12.0 and Ca²⁺ (1.0-5.0 mM) is required for activation. Additionally, ProK maintains its activity in the presence of detergents often used in an assay. Immobilization of an enzyme increases its durability under harsh conditions and simplifies its removal from the reaction medium before the rest of the assay is completed.

Proteinase K may be used to treat a sample as the sample contacts, for example, the sample inlet, a sample outlet, a flow channel, a nitrocellulose membrane, or another surface on which the sample may flow. ProK also may be embedded within the layers of the microfluidic device. Exemplary methods for using ProK are described in the Example 2.

In other embodiments, the ProK is adsorbed to a porous material (e.g., Whatman paper) and inserted into the initial test sample prior to depositing the initial test sample into the sample inlet of the microfluidic device or the ProK may be adsorbed to a small strip of porous material and placed in the sample inlet before, concurrently, or after the test sample. Treatment of the initial sample with ProK or by lining a surface with ProK may be advantageous by reducing interaction of proteins with the surfaces of the microfluidic device and by opening up an epitope of the analyte to permit better antibody binding, thereby increasing the sensitivity of the device. The microfluidic device may include ProK disposed on one or more surfaces even when the sample is pre-treated with ProK as described above.

In some embodiments, the concentration of ProK applied to a surface or other aspect of a microfluidic device is about 0.5 μg/mL to about 1000 μg/mL, about 1 μg/mL to about 950 μg/mL, about 5 μg/mL to about 900 μg/mL, about 10 μg/mL to about 800 μg/mL, about 15 μg/mL to about 700 μg/mL, about 30 μg/mL to about 650 μg/mL, about 50 μg/mL to about 600 μg/mL, about 100 μg/mL to about 550 μg/mL, or about 250 μg/mL to about 500 μg/mL. In one embodiment, the concentration of ProK applied to a surface or other aspect of a microfluidic device is about 400 μg/mL.

In some embodiments, the target analyte is one or more of a protein, a peptide, an amino acid, a nucleic acid, a carbohydrate, a hormone, a steroid, a vitamin, a drug, a pollutant, or a pesticide. In other embodiments, the analyte is one or more of a protein, a peptide, an amino acids, a nucleic acid, a lipid, a carbohydrate, a liposaccharide, or an organic compound derived from a bacterial pathogen, viral pathogen, or fungal pathogen. In other embodiments, the analyte is derived from a protozoan pathogen or a multi-cellular parasitic pathogen, allergen, or a tumor. In other embodiments, the analyte is an antigen derived from a bacterial pathogen, viral pathogen, fungal pathogen, a protozoan pathogen or a multi-cellular parasitic pathogen, an allergen, a tumor, or a mammalian cell.

In certain embodiments, the analytes are derived from a viral pathogen. Exemplary viral pathogens include, e.g., respiratory syncytial virus (RSV), hepatitis B virus (HBV), hepatitis C virus (HCV), Dengue virus, herpes simplex virus (HSV; e.g., HSV-I, HSV-II), molluscum contagiosum virus, vaccinia virus, variola virus, lentivirus, human immunodeficiency virus (HIV), human papilloma virus (HPV), cytomegalovirus (CMV), varicella zoster virus (VZV), rhinovirus, enterovirus, adenovirus, coronavirus (e.g., SARS), influenza virus (flu), para-influenza virus, mumps virus, measles virus, papovavirus, hepadnavirus, flavivirus, retrovirus, arenavirus (e.g., Lymphocytic Choriomeningitis Virus, Junin virus, Machupo virus, Guanarito virus, or Lassa virus), norovirus, yellow fever virus, rabies virus, filovirus (e.g., Ebola virus or marbug virus), hepatitis C virus, hepatitis B virus, hepatitis A virus, Morbilliviruses (e.g., measles virus), Rubulaviruses (e.g., mumps virus), Rubiviruses (e.g., rubella virus), bovine viral diarrhea virus. For example, the antigen can be CMV glycoprotein gH, or gL; Parvovirus; HIV glycoprotein gp120 or gp140, HIV p55 gag, pol; or RSV-F antigen, etc.

In some embodiments the analytes are derived from a parasite from the Plasmodium genus, such as P. falciparum, P. vivax, P. malariae or P. ovale. Thus, the invention may be used for immunising against malaria. In some embodiments the first and second antigens are derived from a parasite from the Caligidae family, particularly those from the Lepeophtheirus and Caligus genera e.g., sea lice such as Lepeophtheirus salmonis or Caligus rogercresseyi.

In certain embodiments, the analytes are derived from a bacterial pathogen. Exemplary bacterial pathogens include, e.g., Neisseria spp, including N. gonorrhea and N. meningitides; Streptococcus spp, including S. pneumoniae, S. pyogenes, S. agalactiae, S. mutans; Haemophilus spp, including H. influenzae type B, non-typeable H. influenzae, H. ducreyi; Moraxella spp, including M. catarrhalis, also known as Branhamella catarrhalis; Bordetella spp, including B. pertussis, B. parapertussis and B. bronchiseptica; Mycobacterium spp., including M. tuberculosis, M. bovis, M. leprae, M. avium, M. paratuberculosis, M. smegmatis; Legionella spp, including L. pneumophila; Escherichia spp, including enterotoxic E. coli, enterohemorragic E. coli, enteropathogenic E. coli; Vibrio spp, including V. cholera, Shigella spp, including S. sonnei, S. dysenteriae, S. flexnerii; Yersinia spp, including Y. enterocolitica, Y. pestis, Y. pseudotuberculosis, Campylobacter spp, including C. jejuni and C. coli; Salmonella spp, including S. typhi, S. paratyphi, S. choleraesuis, S. enteritidis; Listeria spp., including L. monocytogenes; Helicobacter spp, including H. pylori; Pseudomonas spp, including P. aeruginosa, Staphylococcus spp., including S. aureus, S. epidermidis; Enterococcus spp., including E. faecalis, E. faecium; Clostridium spp., including C. tetani, C. botulinum, C. difficile; Bacillus spp., including B. anthracis; Corynebacterium spp., including C. diphtheriae; Borrelia spp., including B. burgdorferi, B. garinii, B. afzelii, B. andersonii, B. hermsii; Ehrlichia spp., including E. equi and the agent of the Human Granulocytic Ehrlichiosis; Rickettsia spp, including R. rickettsii; Chlamydia spp., including C. trachomatis, C. neumoniae, C. psittaci; Leptsira spp., including L. interrogans; Treponema spp., including T. pallidum, T. denticola, T. hyodysenteriae.

In certain embodiments, the analytes are derived from a fungal pathogen (e.g., a yeast or mold pathogen). Exemplary fungal pathogens include, e.g., Aspergillus fumigatus, A. flavus, A. niger, A. terreus, A. nidulans, Coccidioides immitis, Coccidioides posadasii, Cryptococcus neoformans, Histoplasma capsulatum, Candida albicans, and Pneumocystis jirovecii.

In certain embodiments, the analytes are derived from a protozoan pathogen. Exemplary protozoan pathogens include, e.g., Toxoplasma gondii and Strongyloides stercoralis.

In certain embodiments, the analytes are derived from a multi-cellular parasitic pathogen. Exemplary multicellular parasitic pathogens include, e.g., trematodes (flukes), cestodes (tapeworms), nematodes (roundworms), and arthropods.

In some embodiments, the analytes are derived from an allergen, such as pollen allergens (tree-, herb, weed-, and grass pollen allergens); insect or arachnid allergens (inhalant, saliva and venom allergens, e.g., mite allergens, cockroach and midges allergens, hymenopthera venom allergens); animal hair and dandruff allergens (from e.g. dog, cat, horse, rat, mouse, etc.); and food allergens (e.g. a gliadin). Important pollen allergens from trees, grasses and herbs are such originating from the taxonomic orders of Fagales, Oleales, Pinales and platanaceae including, but not limited to, birch (Betula), alder (Alnus), hazel (Corylus), hornbeam (Carpinus) and olive (Olea), cedar (Cryptomeria and Juniperus), plane tree (Platanus), the order of Poales including grasses of the genera Lolium, Phleum, Poa, Cynodon, Dactylis, Holcus, Phalaris, Secale, and Sorghum, the orders of Asterales and Urticales including herbs of the genera Ambrosia, Artemisia, and Parietaria. Other important inhalation allergens are those from house dust mites of the genus Dermatophagoides and Euroglyphus, storage mite e.g. Lepidoglyphys, Glycyphagus and Tyrophagus, those from cockroaches, midges and fleas e.g., Blatella, Periplaneta, Chironomus and Ctenocepphalides, and those from mammals such as cat, dog and horse, venom allergens including such originating from stinging or biting insects such as those from the taxonomic order of Hymenoptera including bees (Apidae), wasps (Vespidea), and ants (Formicoidae).

In some embodiments, the analytes are derived from a tumor antigen selected from: (a) cancer-testis antigens such as NY-ESO-1, SSX2, SCP1 as well as RAGE, BAGE, GAGE and MAGE family polypeptides, for example, GAGE-1, GAGE-2, MAGE-1, MAGE-2, MAGE-3, MAGE-4, MAGE-5, MAGE-6, and MAGE-12 (which can be used, for example, to address melanoma, lung, head and neck, NSCLC, breast, gastrointestinal, and bladder tumors; (b) mutated antigens, for example, p53 (associated with various solid tumors, e.g., colorectal, lung, head and neck cancer), p21/Ras (associated with, e.g., melanoma, pancreatic cancer and colorectal cancer), CDK4 (associated with, e.g., melanoma), MUM1 (associated with, e.g., melanoma), caspase-8 (associated with, e.g., head and neck cancer), CIA 0205 (associated with, e.g., bladder cancer), HLA-A2-R1701, beta catenin (associated with, e.g., melanoma), TCR (associated with, e.g., T-cell non-Hodgkins lymphoma), BCR-abl (associated with, e.g., chronic myelogenous leukemia), triosephosphate isomerase, KIA 0205, CDC-27, and LDLR-FUT; (c) over-expressed antigens, for example, Galectin 4 (associated with, e.g., colorectal cancer), Galectin 9 (associated with, e.g., Hodgkin's disease), proteinase 3 (associated with, e.g., chronic myelogenous leukemia), WT 1 (associated with, e.g., various leukemias), carbonic anhydrase (associated with, e.g., renal cancer), aldolase A (associated with, e.g., lung cancer), PRAME (associated with, e.g., melanoma), HER-2/neu (associated with, e.g., breast, colon, lung and ovarian cancer), mammaglobin, alpha-fetoprotein (associated with, e.g., hepatoma), KSA (associated with, e.g., colorectal cancer), gastrin (associated with, e.g., pancreatic and gastric cancer), telomerase catalytic protein, MUC-1 (associated with, e.g., breast and ovarian cancer), G-250 (associated with, e.g., renal cell carcinoma), p53 (associated with, e.g., breast, colon cancer), and carcinoembryonic antigen (associated with, e.g., breast cancer, lung cancer, and cancers of the gastrointestinal tract such as colorectal cancer); (d) shared antigens, for example, melanoma-melanocyte differentiation antigens such as MART-1/Melan A, gp100, MC1R, melanocyte-stimulating hormone receptor, tyrosinase, tyrosinase related protein-1/TRP1 and tyrosinase related protein-2/TRP2 (associated with, e.g., melanoma); (e) prostate associated antigens such as PAP, PSA, PSMA, PSH-P1, PSM-P1, PSM-P2, associated with e.g., prostate cancer; (f) immunoglobulin idiotypes (associated with myeloma and B cell lymphomas, for example). In certain embodiments, tumor immunogens include, but are not limited to, p15, Hom/Mel-40, H-Ras, E2A-PRL, H4-RET, IGH-IGK, MYL-RAR, Epstein Barr virus antigens, EBNA, human papillomavirus (HPV) antigens, including E6 and E7, hepatitis B and C virus antigens, human T-cell lymphotropic virus antigens, TSP-180, p185erbB2, p180erbB-3, c-met, mn-23H1, TAG-72-4, CA 19-9, CA 72-4, CAM 17.1, NuMa, K-ras, p16, TAGE, PSCA, CT7, 43-9F, 5T4, 791 Tgp72, beta-HCG, BCA225, BTAA, CA 125, CA 15-3 (CA 27.29\BCAA), CA 195, CA 242, CA-50, CAM43, CD68\KP1, CO-029, FGF-5, Ga733 (EpCAM), HTgp-175, M344, MA-50, MG7-Ag, MOV18, NB/70K, NY-CO-1, RCAS1, SDCCAG16, TA-90 (Mac-2 binding protein/cyclophilin C-associated protein), TAAL6, TAG72, TLP, TPS, and the like.

In some embodiments, the analyte is a nucleic acid (e.g., DNA, RNA such as from the ribosomal 16S gene), a lipid, a liposaccharide, or a carbohydrate derived from a bacterial pathogen, a viral pathogen, or a fungal pathogen. In some embodiments, the analyte has a molecular weight of about 0.1 kDa to about 35 kDa, about 0.5 kDa to about 35 kDa, about 1 kDa to about 30 kDa, about 2 kDa to about 25 kDa, about 5 kDa to about 20 kDa, or about 10 kDa to about 15 kDa. In another embodiment, the analyte has a molecular weight of about 1 kDa to about 15 kDa, about lk Da to about 10 kDa, about 1 kDa to about 5 kDa, or about 1 kDa to about 2.5 kDa. In another embodiments, the analyte has a molecular weight of about 0.5 kDa to about 3 kDa, or about 0.5 kDa to about 1.5 kDa.

In one embodiment, the analyte is lipoarabinomannan from Mycobacterium tuberculosis (M. tuberculosis). Lipoarabinomannan (LAM) is a surface glycolipid and major structural component of the M. tuberculosis cell wall and an important mediator of functions that promote productive infection and pathogenicity. LAM contains four distinct structural domains: (1) a phosphatidylinositol anchor, (2) an a-(1→6)-linked mannan backbone of mannopyranose (Manp) residues with pendant a-(1→2)-Manp-linked side chains, (3) an arabinan chain containing multiple arabinofuranoside (Araf) residues with tetra- and hexa-Araf termini, and (4) terminal caps containing various carbohydrate motifs. (See, for example, Chatterjee et al., Glycobiology, Volume 8, Issue 2, February 1998, p. 113-120).

In another embodiment, the analyte is the Ag85 complex of Mycobacterium tuberculosis that comprises the three protein subunits Ag85A, Ag85B,and Ag85C. In one embodiment, the analyte is one or more of Ag85A, Ag85B,and Ag85C.

In some embodiments, the target analyte is found in a test sample such as blood, serum, plasma, buffy coat, wound exudates, pus, lung and other respiratory aspirates, nasal aspirates, bronchial lavage fluids, saliva, sputum, medial and inner ear aspirates, cyst aspirates, cerebral spinal fluid, feces, urine, tears, mammary secretions, ovarian contents, ascites fluid, mucous, stomach fluid, gastrointestinal contents, urethral discharge, synovial fluid, peritoneal fluid, vaginal fluid or discharge, amniotic fluid, semen or the like.

In one embodiment, the test sample is a urine sample, and the analyte is lipoarabinomannan from Mycobacterium tuberculosis.

An exemplary embodiment of a microfluidic device and method of use is shown in FIG. 1 and FIG. 2. With reference to FIG. 1a and 1d, an exemplary microfluidic device generally comprises a sample inlet area 2 fluidly connected to a first sample outlet 4 and a first flow channel 6. The first sample outlet 4 intersects with the nitrocellulose test zone 10. Test zone 10 comprises a proximal end 12, a center region 14, and a distal end 16. The distal end 16 of the test zone 10 is fluidly connected to an absorbent pad 18. The proximal end 12 of the test zone is fluidly connected with flow channel 6 via a second sample outlet 8. FIG. 1b shows the construction of an exemplary microfluidic device comprising a laminate of one or more double-sided adhesive film 24 disposed between layers of the hydrophilic polyester film 22. The depths, widths, and size of the various channels, sample inlets, sample outlets, etc. may be adjusted by varying the adhesive film layers of the laminates and cutting the final design with a cutting apparatus such as a laser. FIG. 1c shows an exemplary testing zone 10 comprising a distal end 16, center region 14, and proximal end 12. The area between the center region 14 and distal end 16/absorbent pad 18 comprises the antibody zone 20. Antibody zone 20 comprises at least three distinct regions: a detection zone 26, a capture zone 28, and a control zone 30, each of the zones having one or more means of detection an analyte dried on a surface of the antibody zone 20 (i.e., nitrocellulose membrane). For example, the detection zone comprises one or more mobilizable analyte detection antibodies conjugated to a labeling component 38. The mobilizable detection antibody 38 may be rehydrated and desorbed from the surface of the antibody zone 20 upon contact with the test sample as it flows over the antibody zone 20. The capture zone 28 comprises one or more capture antibodies 40 that specifically bind to the target analyte. The capture antibodies are immobilized to the surface of the antibody zone 20. If the target analyte is present in the test sample, the capture antibodies 40 may specifically bind to the analyte after the analyte is specifically bound by the mobilizable detection antibody (detection antibody+analyte complex) 38. Any desorbed and unbound detection antibody 38 may specifically bind to one or more control antibodies 42 that are immobilized in the control zone 30 and configured to specifically bind to the mobilizable detection antibody (i.e., an anti-detection antibody antibody) 38.

FIG. 2a-c depict the use of an exemplary microfluidic device. A test sample 36 comprising, for example, a bodily fluid having or suspected of having one or more target analytes, is deposited in sample inlet 2. The test sample 36 migrates through the first sample outlet 4 and through the first flow channel 6 to the second sample outlet 8 via capillary action. A first fraction of the test sample that exits the first sample outlet 4 and intersects with the center region 14 of the testing zone 10. The first test sample fraction then flows towards both the proximal end 12 and the distal end 16 of the testing zone 10. A second test sample fraction migrates through both the first flow channel 6 and second sample outlet 8 to arrive at the proximal end 12 of the testing zone 10. The second test sample fraction arrives at the proximal end 12 after the first test sample fraction has arrived at the center region 14 and is spread through the testing zone 10. The second test sample fraction arrives at the testing zone 10 after the first test sample fraction because of the greater distance of travel of the second test sample fraction due to the length of the first sample outlet being shorter in length than the length of the first flow channel 6 and second sample outlet 8.

As the first test sample fraction flows towards the distal end 16 and absorbent pad 18, the first test sample fraction flow over the antibody zone 10 where the first test sample fraction rehydrates and desorbs the mobilizable detection antibody 38 dried to the surface of the detection zone 26. Once rehydrated and desorbed from the detection zone 26, the mobilizable detection antibody 38 is mixed with the test sample and is free to specifically bind to the target analyte to form a complex (detection antibody+analyte). The complex now migrates through the capture zone 28 where one more capture antibodies 40 are immobilized to the surface of the capture zone 28. The capture antibody 40 may then specifically bind to the analyte in the complex. Excess desorbed and unbound (i.e., not bound to a target analyte) detection antibody 38 flows through the control zone 30 comprising one or more control antibodies 42 immobilized to the surface of the control zone 30. The control antibodies 42 specifically bind to the desorbed and unbound detection antibodies (i.e., anti-detection antibodies) 38.

After the first test sample fraction has arrived at the center region 14, the second test sample fraction flows from the second sample outlet 8 towards the absorbent pad 18. As the second test sample fraction passes over proximal end 12, the second test sample fraction may rehydrate one or more reagents 32, 34 dried to the surface of the proximal end 12. The reagents 32, 34 may include labeling components, colorimetric components, buffers, or other reagents useful for generating a detectable signal from the signal component conjugated to the mobilizable detection antibody. In the present example, one dried reagent is a colorimetric substrate 32 (e.g., DAB) and a second reagent is hydrogen peroxide 34. Next, the first and second test sample fractions mix and transport the rehydrated reagents 32, 34 over the capture zone 28 and control zone 30 of antibody zone 20. If the target analyte is present in the test sample, then the complex will specifically bind to the capture antibody 40 and any desorbed and unbound detection antibody 38 will specifically bind to the anti-detection antibodies 42, thereby producing two detectable signals (one detectable signal 46 in the capture zone 28 and one detectable signal 44 in the control zone 30). If no analyte is present, then the mobilizable detection antibody 38 will only specifically bind with the anti-detection antibody 42 and produce a single detectable signal 44 in the control zone.

Accordingly, in one embodiment, a microfluidic device comprises a testing zone comprising a nitrocellulose membrane comprising a proximal end, a distal end, and a center region, wherein the testing zone comprises an antibody zone disposed between the distal end and the center region of the testing zone, wherein the antibody zone comprises, in order from the center region to the distal end: a detection zone comprising mobilizable detection antibodies conjugated to a labeling component and spot-dried to a surface of the detection zone; a capture zone comprising one or more capture antibodies that are spot-dried and immobilized on a surface of the capture zone; and a control zone comprising one or more anti-mobilizable detection antibodies that are spot-dried and immobilized on a surface of the control zone; a substrate component and hydrogen peroxide separately spot-dried on a surface of the testing zone between the proximal end of the testing zone and the center region of the testing zone; a sample inlet for receiving a sample comprising: a first sample outlet intersecting with, and in fluid communication with, the center region of the testing zone; a second sample outlet fluidly connected to a first flow channel, wherein the first flow channel is in fluid communication with the proximal end of the testing zone; and an absorbent pad in fluid communication with the distal end of the testing zone; wherein the first flow channel has a greater length than the length of the first sample outlet.

In some embodiments, a second flow channel is disposed between the first sample outlet and the center region of the testing zone, wherein the length of the first flow channel is greater than a combined length of the first sample outlet and the second flow channel.

The disclosure also provides for methods of detecting certain analytes using a device as described herein. In one embodiment, a method of detecting a target analyte in a test sample comprising: a) contacting a microfluidic device as described herein with the test sample comprising one or more target analytes and one or more buffer components, wherein the test sample is received in the sample inlet, wherein a first fraction of the test sample migrates by capillary action through the first sample outlet to contact the center region of the testing zone, wherein the first fraction of the test sample flows toward both the proximal end of the testing zone and the distal end of the testing zone, wherein the first fraction of the testing sample rehydrates, mixes, and spreads the desorbed mobilizable detection antibody conjugated to a labeling component over the antibody zone; b) binding the desorbed mobilizable detection antibody to the one or more target analytes to form an analyte-antibody complex, wherein the analyte-antibody complex then binds to the immobilized capture antibody, and the immobilized anti-detection antibody specifically binds to desorbed and unbound mobilizable detection antibody; c) migrating, by capillary action, the second fraction of the test sample through the flow channel towards the distal end of the testing zone such that the second fraction rehydrates, spreads, and the mixes the substrate component and the hydrogen peroxide over the testing zone; d) detecting a signal from the analyte-antibody complex bound to the immobilized capture antibody, the desorbed and unbound mobilizable detection antibody attached to the immobilized anti-detection antibody, or a combination thereof, wherein a detectable signal from both the analyte-antibody complex bound to the immobilized capture antibody and the desorbed and unbound mobilizable detection antibody attached to the immobilized anti-detection antibody indicates the presence of the target analyte in the test sample.

In another embodiment, a method of determining the presence or absence of a target analyte in a test sample comprising contacting a microfluidic device as described herein with a sample; forming a complex comprising the target analyte specifically bound to the mobilizable detection antibody; and measuring a detectable signal produced by: a) both the complex specifically bound to the immobilized capture antibody and the mobilizable detection antibody not attached to the complex that specifically binds to the immobilized anti-detection antibody; or b) the mobilizable detection antibody not attached to the complex that specifically binds to the immobilized anti-detection antibody; thereby determining the presence of the target analyte in the test sample if the detectable signal is produced as recited in part a) and the absence of the target analyte in the test sample if the detectable signal is produced as recited in part b).

In some embodiments, the initial test sample of bodily fluid is about 20 μl to about 150 μl in volume, about 30 μl to about 120 μl in volume, about 40 μl to about 100 μl in volume, about 50 μl to about 95 μl in volume, about 60 μl to about 95 μl in volume, about 70 μl to about 95 μl in volume, about 80 μl to about 95 μl in volume, or about 85 μl to about 95 μl in volume. In some embodiments, the initial test sample of bodily fluid is about 75 μl to about 95 μl in volume, or about 80 μl to about 90 μl in volume, or about 80 μl to about 85 μl in volume.

### Results and Discussion

### Flow Characteristics in the Device

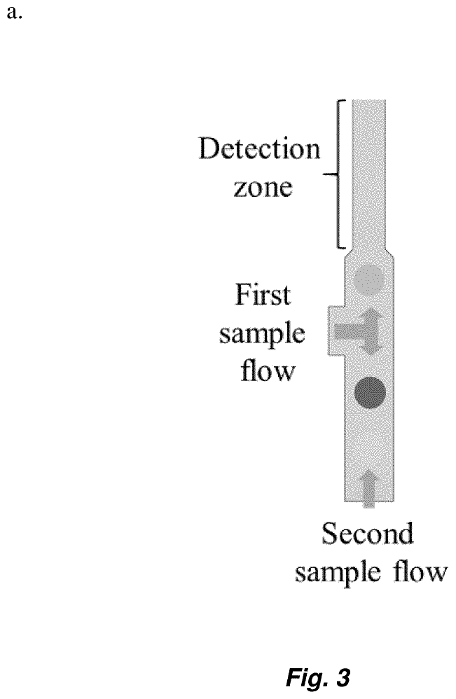

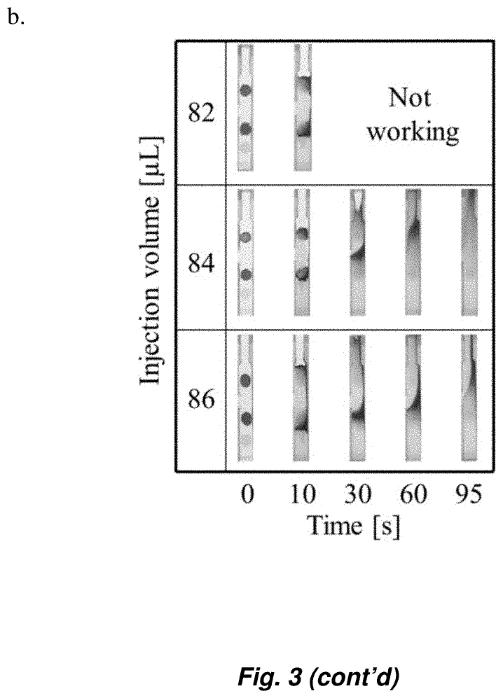

Before testing real samples, the flow characteristics of the device were tested using dye solutions. Various dyes were spotted on the nitrocellulose membrane to represent detection Ab, DAB substrate, and H₂O₂, respectively (FIG. 3a). Once the sample was loaded in the sample inlet, it split into channel 1 and channel 2. The capillary action creates a pressure drop at the flow front and generates flow through the channel. The pressure gradient within the channel by capillary action is small compared to traditional pressure-driven microfluidics. Therefore, the sample was transported to each outlet without flow problems such as leaking. Since outlet 1 was very close to the inlet area, the sample solution reached the nitrocellulose membrane faster than the flow from outlet 2. The solution from outlet 1 rehydrated the entire nitrocellulose strip, flowing in both directions from the outlet, before the solution from outlet 2 reached the nitrocellulose (FIG. 3a). Even if the solution from channel 2 reached the nitrocellulose membrane before the nitrocellulose was fully saturated by channel 1, most of the sample continued to flow through channel 1 because of the close proximity of outlet 1 to the nitrocellulose. As a result, the dye was transported over the capture strip while the dye was delayed, preventing the mixing between detection Ab and DAB substrate in a functional device (FIG. 3b). During the delay between the detection Ab and DAB, sample from outlet 1 washed all excess detection antibody from the detection zone. The washing that occurred during the delay ensured that excess enzyme label and substrate do not interact, which would cause a large background signal. Once liquid in the sample inlet was depleted, the remaining liquid in channel 1 and channel 2 began to flow through the nitrocellulose membrane and into the absorbent pad pump. The order of flow from each channel depended on the length of the channel, so the liquid in channel 2 was delayed again until channel 1 was empty. As a result, the device was able to sequentially deliver dye, wash buffer, and a mixture of dyes to the detection zone with a single injection step.

The sample volume flowing through outlets 1 and 2 depended on the injection volume and the channel length. FIG. 3b shows the flow of dyes as a function of three different injection volumes. At 82 μL, the sample solution did not fill the channels fully. Above 82 μL the channels were fully filled, and sequential delivery was achieved (FIG. 3b). As sample volume increases from 84 to 86 μL, the volume of wash buffer between the dyes also increased, which was visible at the 30 s time mark in FIG. 3b. Unfortunately, increasing the volume from 84 to 86 μL also impacted the flow pattern. At 86 μL, sample was flowing from outlet 1 and 2 simultaneously so the dye (substrate) was forced into the right side of the nitrocellulose. Because uneven substrate flow over the test line may result in lower sensitivity, an 84 μL sample volume was chosen for this assay. Alternatively, if a larger or smaller sample volume is desired, the length of channel 2 or the design of nitrocellulose membrane could be changed to compensate for flows. If the size of the fluidic device and nitrocellulose membrane is changed, the minimum loading volume should be re-optimized before the assay is performed.

### Assay Optimization

Next, various parameters were optimized such as the amount of PVP, Triton X-100, substrate, the concentration of capture Ab and HRP-conjugated detection Ab to achieve the highest assay performance. All optimization experiments used 1 μg/mL LAM in sample solution.

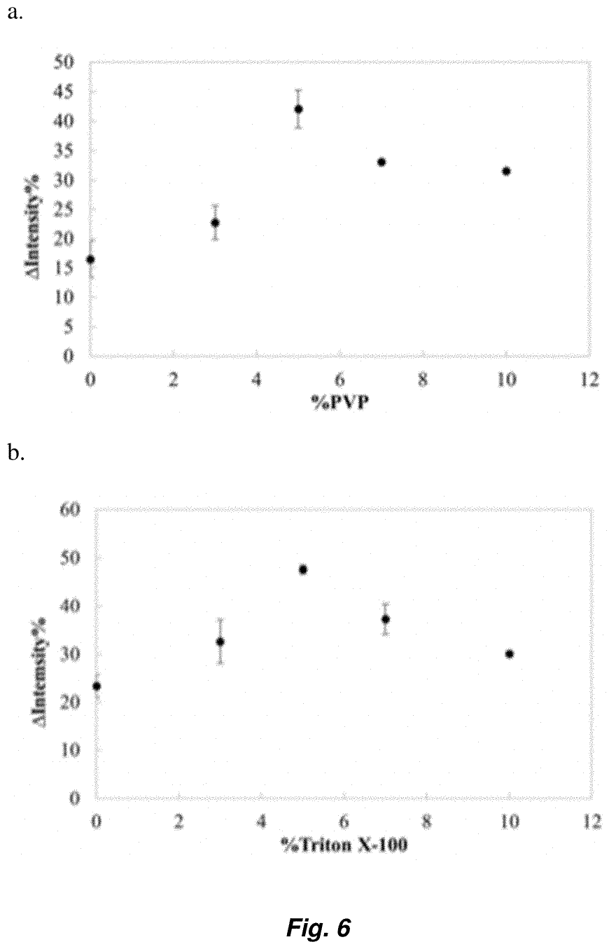

1) Concentration of PVP and Triton X-100. Since the detection Abs are directly spotted on the nitrocellulose membrane and delivered through a detection zone afterward. PVA and Triton X-100 were applied to minimize permanent immobilized of detection Ab on the nitrocellulose membrane. PVP and Triton X-100 in the detection Ab drying buffer are critical to minimize protein absorption and nonspecific protein-protein interaction, respectively. First, the Triton X-100 was fixed at 2% and the detection Ab concentration at 50 μg/mL and tested five different PVP concentrations of 0, 3, 5, 7, and 10%. FIG. 6a shows that 5% PVP provides the highest ΔI %. After optimizing PVP concentration, five different Triton X-100 concentrations of 0, 3, 5, 7, and 10%. were tested. FIG. 6b indicates that 5% Triton X-100 provides the highest ΔI %. A drying buffer with 5% PVP and 5% Triton X-100 was used in all remaining experiments.

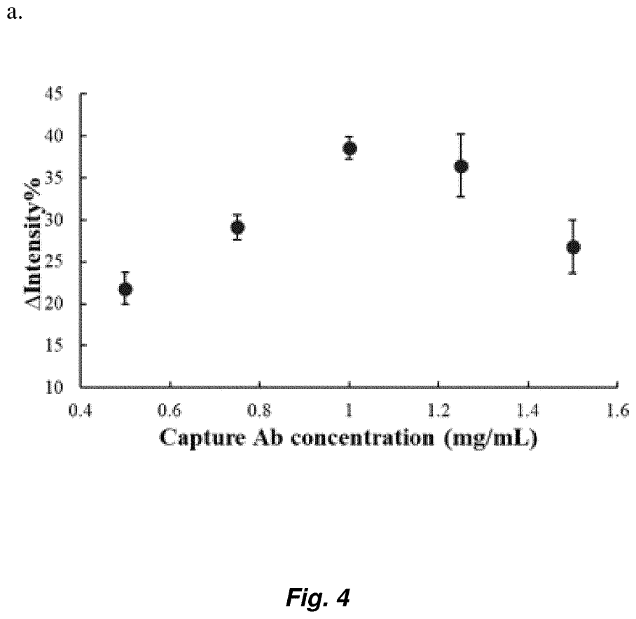

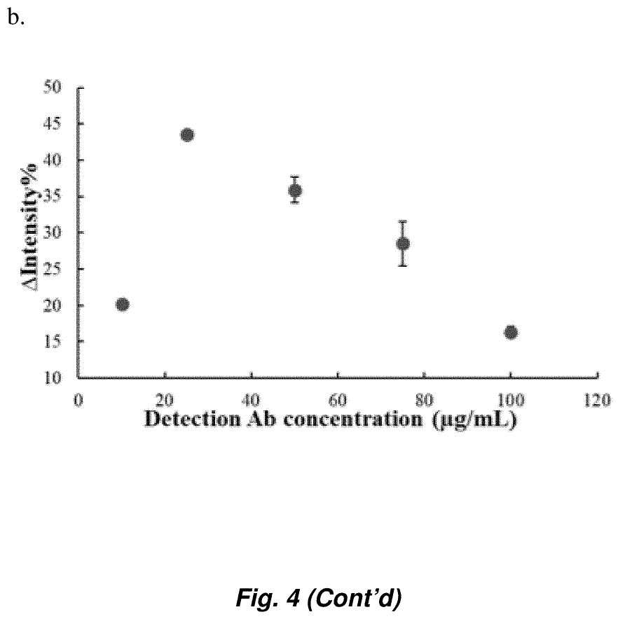

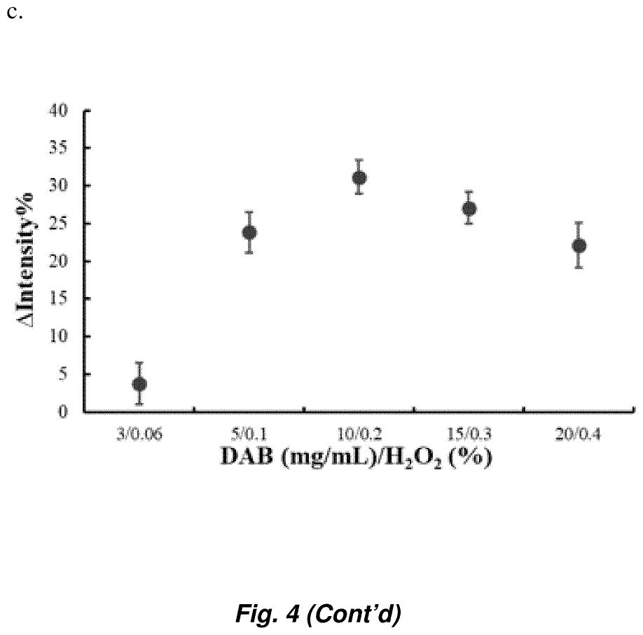

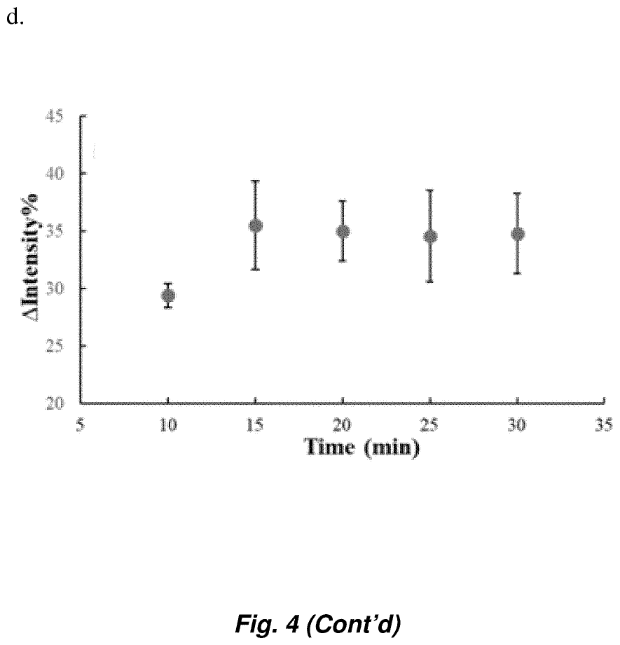

2) Concentration of capture Ab and detection Ab. To obtain the optimum amount of capture Ab, the detection Ab was fixed at 50 μg/mL in this assay. 0.2 μL of capture Ab was immobilized at the test zone with concentrations of 0.5, 0.75, 1, 1.25, and 1.5 mg/mL. 1 mg/mL gave the highest intensity and was used as the capture Ab concentration for remaining experiments (FIG. 4a). Detection Ab concentration was optimized in FIG. 4b. The highest signal intensity was achieved at 25 μg/mL of detection Ab, so this concentration was used in all remaining experiments. Higher concentrations of capture and detection antibody resulted in higher background signal, so it would be possible to increase antibody concentration with a more efficient washing step.

3) Substrate concentration. The recommended ratio of DAB:H₂O₂ from the manufacturer is 1 mg/mL:0.02%. In this experiment that ratio is kept constant but the total amount of DAB and H₂O₂ were varied. Results in FIG. 4c show maximum signal was achieved with 10 mg/mL DAB and 0.2% H₂O_{2.} Above these concentrations the signal intensity decreased because of large background.

4) Assay time. As a POCT solution, assay time is an important parameter to consider. Flow time in the device is dictated by the total sample volume, which is fixed based on the device geometry. Signal is generated in 10 min, but intensity increases as a function of time due to a longer interaction between the substrate and enzyme label. FIG. 4d shows the signal intensity at five timepoints. After 15 min, the signal intensity plateaus. To minimize assay time and maximize signal 15 min was chosen as the wait time between loading sample and reading the result.

### Analytical Performance of the Device

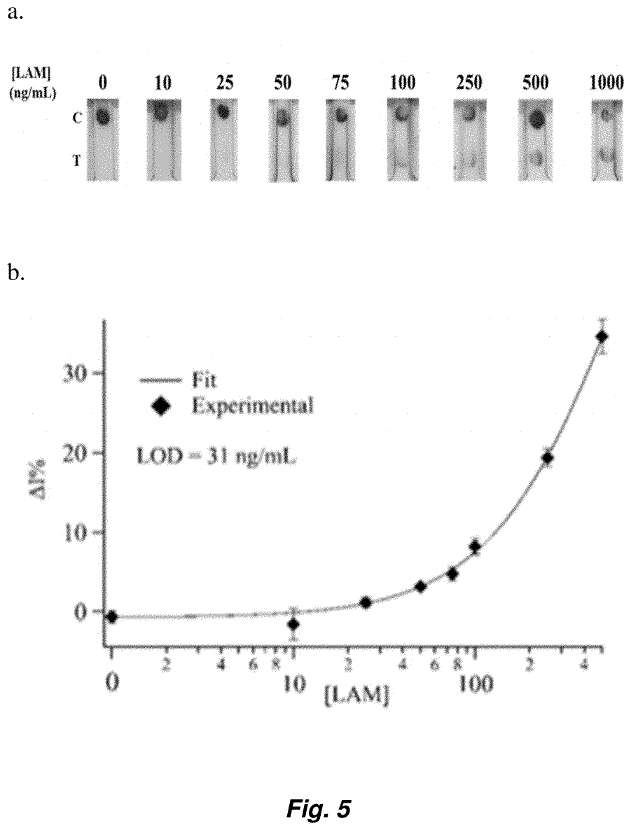

The analytical performance for LAM detection using the microfluidic interface device described in this work was studied using the optimal parameters found above. LAM was tested in the range of 10-1000 ng/mL. The color change was confirmed by naked eye at a minimum of 25 ng/mL (FIG. 5a). For semi-quantitative analysis (FIG. 5b), signal vs LAM concentrations were fit to a 4-parameter logistic curve commonly used for sandwich immunoassays (Equation 2).

$$f ( x ) = d + \frac{a - d}{1 + {( \frac{x}{c} )}^{b}} \qquad ( Eq . 2 )$$

where f(x) is the signal, x is the target concentration, a is the expected response at x=0, b is the slope of the curve at point c , which is the target concentration corresponding to f(x)=(a+d)/2x, and d is the expected response when the target concentration is infinitely high.

Using 3×σ₀ as the signal for the lower limit of detection (LOD), the detection limit was calculated as 31 ng/mL. The quantitative detection limit is higher than the by-eye reading because of background signal in the blank and large standard deviation in the 10 ng/mL data point (FIG. 5b). Table 1 shows a comparison of the analytical performance of the disclosed devices against conventional ELISA and LFIA systems. The LOD of the device is higher than other methods and commercial LFIAs such as AlereLAM and FujiLAMClick or tap here to enter text. Although ELISAs can provide low LODs, they require many pipetting steps and lengthy incubation times, typically 1-2 hours or more, for antibody binding with the analyte. As a result, analysis times are several hours. Similarly, the FujiLAM device can provide better LOD because the antibody in the LFIA device is incubated for 40 min with sample solutions. The system also uses a silver enhancement step to increase signal intensity. As shown in Table 1, methods with good sensitivity typically require long analysis times.

TABLE 1
Analytical performance of conventional ELISA
and LFIA platforms for LAM detection.

| | | | | Analysis | |
|---|---|---|---|---|---|
| Platform | Signaling agent | LOD | | time | |
| Conventional | HRP-TMB | 0.1 | ng/mL | >5 | hrs |
| ELISA | | | | | |
| Conventional | HRP-TMB | 0.05 | ng/mL | >5 | hrs |
| ELISA | | | | | |
| LFIA | AuNPs | 0.5 | ng/mL | 25 | min |
| (AlereLAM) | | | | | |
| LFIA | Silver enhanced | ~0.010-0.02 | ng/mL | 50-60 | min |
| (FujiLAM) | AuNPs | | | | |
| Sequential | | | | | |
| delivery | HRP-DAB | 25 | ng/mL | 15 | min |
| microfluidic | | | | | |
| interface LFIA | | | | | |

According to prior publications, enzyme-based LFIAs enhance the sensitivity compared to AuNPs-based LFIAs. However, the AlereLAM device which uses AuNPs provided lower LOD than the disclosed device because of activity of antibodies since affinity binding of antibodies is one of factors affecting the assay performance. Therefore, employing a new pair of antibodies should be considered to improve sensitivity. As shown in Table 2, the enzyme based LFIA system has been used in the conventional lateral flow assay platform. The publications in Table 2 all take less than 30 min, which is a significant improvement over conventional ELISA. However, multi-step operation (3-4 steps) is still required for sample loading, washing and substrate addition. These steps are manual and timed, so the end-user must be actively observing the assay for the duration of the test, which is unsuitable for being a POCT device.

TABLE 2
Related works of enzyme based LFIA
device using naked eye detection.

| | | Number | Analysis |
|---|---|---|---|
| | | of | time |
| Platform | Enzyme-substrate | operations | (min) |
| Conventional LFIA | HRP^{a}-TMB^{b}, | 4 | >20 |
| | AEC^{c}, DAB^{d} | | |
| Conventional LFA | HRP-AuNP-AEC | 4 | 30 |
| Conventional LFIA | HRP-AuNP-TMB | 4 | <20 |
| Sequential delivery | HRP-DAB | 2 | 15 |
| microfluidic interface | | | |
| LFIA | | | |

^{a}Horseradish Peroxidase
^{b}3,30,5,50-Tetramethylbenzidine
^{c}3-amino-9-ethyl-carbazole
^{d}3,30-Diaminobenzidine tetrahydrochloride

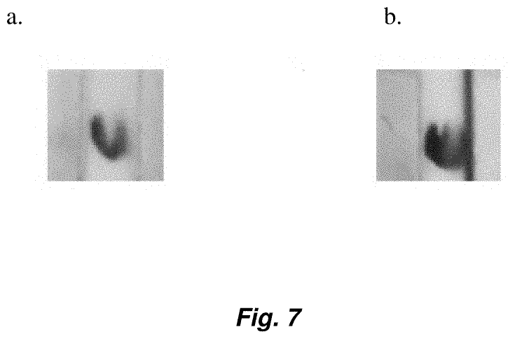

The microfluidic interface developed in this work minimizes the number of manual steps and assay time by automating the delivery of each reagent to the detection zone. In its current form, the device required two end-user steps because adding fresh H₂O₂ improved the signal intensity over dried H₂O₂ (FIG. 7). Future iterations of the device will explore alternate means to stabilize dry H₂O₂ and/or to integrate the H₂O₂ into the sample buffer. Even with two manual steps, the device operation is simple and no timed operations are needed during the assay. In addition, the developed device does not require additional pipetting steps for washing, unlike ELISA and enzyme-based LFIA systems, because sample solution acts as a washing solution after flowing the detection-Ab over the text and control spots. This step could decrease the background noise caused by non-specific binding on the test zone. Therefore, the microfluidic interface minimizes assay time (within 15 min) compared to conventional ELISA and the simplicity enables use at the point-of-care. In situations where additional washing or sensitivity is needed, the length of the fluidic channels and/or the nitrocellulose can be increased. A longer nitrocellulose would increase the gap between substrate and detection zone and therefore increase the washing volume. Lengthening the two channels would increase the volume of sample that would be processed with the device, which would increase the sensitivity, but also assay time. In situations where sample volume is limited, these parameters can be decreased.

### Application in Urine Sample

Urine samples from healthy human volunteers were tested to demonstrate the reliability and feasibility of the proposed device as well as the matrix effect of real samples. Urine samples spiked with LAM at different concentrations were added to the device and the same procedures used in the buffer were followed. LAM spiked in urine samples at 50, 100, and 250 ng/mL were tested, because these are in the range of real urine samples (0.1 ng/mL to hundreds ng/mL) as well as in the range of calibration curve. The results showed analytical recoveries in a range of 100.4%-108.2% with the relative standard deviation (% RSD) ranging from 0.4%-1.1% (Table 3). While normal urine samples were tested, the matrix of healthy urine sample and clinical urine sample will be different. A previous study performed LAM spiked in healthy urine sample and non-TB patients. All samples were pretreated with 200 μg/mL of proteinase K before testing to reduce matrix effect from protein of urine sample as well as opening up the epitope on LAM for antibody binding ability resulting in increased sensitivity. The obtained LOD of LAM spiked in healthy urine sample was similar to non-TB patients. From these results, the proposed device can be used as an alternative POCT device for LAM detection in urine samples or for any other biomarkers present in urine. Furthermore, recently published work has reported single step of urine sample pretreatment by immobilizing proteinase K onto a Whatman paper. This strategy is able to minimize both step and time of sample preparation. Therefore, this would be a promising choice for integration of immobilized proteinase K pad into a capillary-driven device which is more deliverable to end users for further development.

TABLE 3
Recovery testing of urine spiked LAM in different concentrations
performing by microfluidic interface device (n = 3).

| | LAM | | | |
|---|---|---|---|---|
| | concentrations | Measured | | |
| | in urine sample | concentrations | | |
| Sample No. | (ng/mL) | (ng/mL) | % Recovery | % RSD |
| 1 | 50 | 54.10 ± 0.23 | 108.2 | 0.4 |
| 2 | 100 | 102.67 ± 0.75 | 102.7 | 0.7 |
| 3 | 250 | 250.99 ± 2.7 | 100.4 | 1.1 |

A new microfluidic interface platform for on-site enzyme based LFIA analysis was demonstrated. The platform functions through the integration of two different microfluidic materials to automate the ordered flow of sample and immunoreagents over the capture zone on a nitrocellulose membrane. The capillary-driven microfluidic channel fabricated by polyester film and pressure sensitive adhesive was used for sample loading and timed delivery. The nitrocellulose membrane with the spotted reagents was used as the immunoreaction/detection area. As a result, the device sequentially transported the HRP-conjugated detection Ab and its substrate to the detection zone without any additional steps after loading the sample. Sequential delivery was vital to this assay to ensure excess enzyme label did not react with the substrate and cause high background. The LAM was used as an analyte with the intended application of tuberculosis testing in low resource settings. In a buffer system the minimum concentration of color change and achieved LOD (3xao) were found to be 25 ng/mL and 31 ng/mL, respectively. Urine samples were also successfully applied to the device to simulate the sample matrix of choice for LAM detection. The results presented in this manuscript demonstrate that the capillary-driven microfluidic interface platform can be used as an alternative POCT device due to its rapidity, ease of operation, low cost, and potential for mass production. Additionally, more sensitive substrate for HRP, such as 3,3′,5,5′-tetramethylbenzidine (TMB) and/or a more sensitive pair of Ab will be tested with clinical samples in the next generation device development to improve the assay performance and demonstrate clinical validity. Not only more sensitive substrate and/or Ab pair but also the selectivity and shelf-life device testing will be concerned in the further development.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. Material and Methods

Chemicals and materials. Anti-lipoarabinomannan (LAM) monoclonal capture and detection antibodies were obtained from Chatterjee Lab repository. Anti-detection antibody was purchased from Sigma Aldrich. LAM was obtained from the Chatterjee Lab repository that was isolated and purified prior from Mycobacterium tuberculosis (Mtb) CDC1551 strain from in vitro culture. Normal urine (NEU) samples were collected from a healthy human volunteer from a TB non-endemic region. Horseradish peroxidase (HRP) conjugation kit-Lightning-Link (ab102890) was purchased from Abcam. 3,3′-diaminobenzidine tetrahydrochloride (DAB) was ordered from Thermo Scientific. Hydrogen peroxide (H₂O₂), bovine serum albumin (BSA), phosphate buffer saline tablet (PBS), Tween 20, Thimerosal, Ethylenediaminetetraacetic acid (EDTA), and Polyvinylpyrrolidone (PVP; MW 29,000) were obtained from Sigma Aldrich. Triton X-100 and ferrous sulfate (FeSO₄.7H₂O) were purchased from Fisher Scientific. Trehalose was obtained from Calbiochem. Milli-Q (MQ) water was used to prepare all reagents. Nitrocellulose membrane (FF120HP, GE), absorbent pad (AP30034P0, Millipore), diagnostic microfluidic hydrophilic film (9962, 3M), and adhesive transfer tape (468MP, 3M), which has a high chemical resistance, were used to fabricate the device.

Device fabrication and preparation . The microfluidic interface was composed of a capillary-driven flow channel, nitrocellulose membrane, and absorbent pad as shown in FIGS. 1a and 1b. The flow channel consisted of four layers of hydrophilic polyester and double-sided adhesive film. The polyester film was used as bottom and top layers of the flow channel. The channel patterns were cut in the adhesive film layers and were placed between the polyester films. The channel height was controlled by the number of adhesive film layers. To fabricate a 200 μm channel height, two adhesive film sheets were stacked. The fluidic device consists of a single sample inlet (1×1 cm) that has two outlets (outlet 1 and 2) connecting to channels of different lengths. The short channel (channel 1) connects to outlet 1 and has a internal volume of 1.5 μL, while the long channel (channel 2) connects to outlet 2 and has a internal volume of 56.4 μL. The nitrocellulose membrane was cut to fit into the fluidic device such that outlet 1 intersects the middle of the membrane and outlet 2 connects to the end of the membrane. The nitrocellulose membrane is 30 mm long. Above the detection zone, the nitrocellulose membrane/strip is 3 mm wide, but tapers to 2 mm wide at the detection zone. All geometries were designed using CorelDRAW and cut out using a laser cutter with 27% power of vector mode (Zing 10000, Epilog Laser).

FIG. 1c shows the reagent patterning on the nitrocellulose membrane. 0.2 μL of 1 mg/mL of anti-LAM Ab (capture Ab) and anti-detection Ab prepared in 10 mM PBS pH 7.4 were spotted at the test (T) and control (C) zone, respectively and the nitrocellulose membrane was dried at 37° C. then blocked with 1% BSA in 10 mM PBS pH 7.4 for 25 min followed by washing with 0.1% PBS Tween 20 for 3 min. After washing, the nitrocellulose membrane was left in a 37° C. incubator for 1 hr to dry. Next, 5 μL of 25 μg/mL of anti-LAM-HRP Ab (detection Ab) was diluted in drying buffer pH 7.4 containing 5% PVP and 5% Triton X-100. 0.5 μL of 10 mg/mL of DAB in 10 mM PBS pH 7.4 containing 4% trehalose, to extend the shelf-life of DAB, were spotted onto the blocked nitrocellulose membrane followed by drying at 37° C. for 15 min. Immediately before sample addition, 0.5 μL of 0.2% of H₂O₂ in 10 mM PBS pH 7.4 was dropped onto nitrocellulose membrane. FIG. 1d shows a photograph of a completed device.

Urine sample preparation. Urine samples were prepared according to a previous study. LAM was spiked into NEU and stored at 4° C. for 30 min. 200 μg/mL of proteinase K was then added to spiked urine samples and the samples incubated at 55° C. for 30 min . After 30 min, the proteinase K was inactivated by heating at 100° C. for 30 min. The increase in temperature induces denaturation of protein as well as proteinase K and crosslinking of the protein fragments, allowing for easier removal via centrifugation at 12,000×g for 10 min. Click or tap here to enter text. Finally, the supernatant of sample was used for testing.

Assay procedure and data analysis. Once the device is ready to test, 0.5 μL 0.2% of H₂O₂ was added to the nitrocellulose membrane next to outlet 2 followed by 84 μL of sample containing of LAM to the sample inlet. After sample addition, the solution split between channels 1 and 2. The solution from channel 1 flowed directly into the nitrocellulose membrane from outlet 1, while the solution in channel 2 slowly filled towards outlet 2. At outlet 1, the sample wetted the nitrocellulose in both directions, which rehydrated and transported the detection Ab over the test and control (T and C) zones, while simultaneously rehydrating the DAB substrate upstream without flowing to the T and C zones. At the test line, the LAM-detection Ab complex binds to the anti-LAM capture Ab. At the control line, excess detection Ab binds with anti-detection Ab (FIG. 2b). After binding at the test and control line, rehydrated DAB and H₂O₂ from channel 2 flow over the detection zone. If LAM was present, the brown DAB product would appear at the T and C zone, and if LAM was absent the colored product will only appear at the C zone (FIG. 2c). After the assay was completed, images of the nitrocellulose strip were captured with an iPhone7 (Apple) and the color intensity was analyzed with ImageJ (National Institutes of Health). The intensity (ΔI %) was calculated using Equation 1²⁵ to eliminate the effect of the negative control,

ΔI[%]=[(I_{c}−Iₜ)/Iₜ]×100% (Eq. 1)

where I_{c} and Iₜ are the intensity value of the test zone for negative control and positive samples, respectively. Intensities are quantified by converting the image to greyscale before measuring.

### Example 2. Proteinase K Immobilized on a Porous Substrate.

### Proteinase K Immobilization on a Porous Substrate (IPK)

Presently disclosed are embodiments of a facile universally applicable method for ProK immobilization. Optimal amounts needed, time to complete digestion, operation temperature, and stability time on the paper were also determined and were monitored by ELISA.

Whatman no.1 paper was used as a model porous substrate but the methods and discussion below is applicable to other porous substrates used in various microfluidic devices such as microchannels or flow channels, sample inlets, etc. The Whatman paper was excised (3×5 mm) and the —OH groups of the carbohydrates were converted to aldehydes via periodate oxidation using NaIO₄ as an oxidizing agent. LiCl was used to enhance the periodate oxidation efficiency because it makes hydroxyl groups more available to periodate oxidation. ProK was covalently linked to the aldehyde groups formed on the paper to create a reverse Schiff base. The remaining aldehyde groups and the Schiff base were reduced by sodium cyanoborohydride (NaCNBH₃) via reductive amination.

### Optimization of Proteinase K on a Porous Substrate (IPK)

Concentration. To test for the immobilization of ProK on Whatman paper, a BCA assay was performed on the ProK stock tube (0-1000 μg/mL) and the washes from the immobilization steps of strips immobilized with varying concentrations (0-1000 μg/mL) of ProK. All samples were tested in duplicate and plotted against the stock ProK curve. No significant loss of ProK during the immobilization steps was observed. These results indicated that the concentration of ProK immobilized on the strips were as specified (0-1000 μg/mL) and no excess ProK washed out. To optimize the concentration of ProK for assay performance, spiked urine was collected from a healthy volunteer with LAM starting at 1 μg/mL and used strips containing varying concentrations of the enzyme (0-1000 μg/mL) treated for 2 hr at room temperature (at 27° C. in a microplate incubator) and then analyzed using indirect ELISA with CS35 mAb. At concentrations 0 and 50 μg/mL, the OD₄₀₅ values were at or near the background levels. At higher concentrations (100-1000 μg/mL), LAM showed increased binding to the Ab with the lowest background at 400 μg/mL. This also confirms retention of ProK on paper after immobilization.

As a comparative control for IPK, non-endemic urine (NEU) spiked with LAM was simultaneously treated with SPK and used for C-ELISA, the OD₄₅₀ values were similar to what was obtained with the IPK treatment.

Time. Using 400 μg/mL as the optimal concentration on IPK in an indirect ELISA, we set out to optimize the time of pretreatment required. To achieve this, urine spiked with LAM was treated with IPK at 400 μg/mL at a time interval of 0, 30, 60, 120, and 180 mins at room temperature and analyzed by indirect ELISA. We observed the best signal as compared to 0 min at 60 and 120 min concluding that in indirect ELISA, in urine spiked with LAM, IPK best performs at a concentration of 400 μg/ml for 60 min at room temperature.

Temperature. Since SPK is optimized at a higher temperature (55° C.), we needed to optimize the pretreatment temperature for IPK. NEU spiked with LAM was pretreated with IPK at 400 μg/mL for 60 min at room temperature, 37° C. (ProK can be activated at this temperature) and 55° C. We observed that at high temperatures (37° C. & 55° C.), OD₄₀₅ improved with concomitant increase in background. At room temperature however, although the absorbance values were lower, there was very low background. This led us to conclude that at 400 μg/mL with IPK, the optimal urine pretreatment could be done at room temperature for 60 min. Incidentally, background interference is one of the most critical issues in developing sensitive POC assays for TB diagnosis, as the analyte concentration is low in a majority of the population.

C-ELISA. To optimize conditions for the use of IPK in the C-ELISA platform, urine spiked LAM was pretreated with IPK at various concentrations (0-1000 μg/mL) at room temperature for 60 min. We noted that at 400 μg/mL, as we had observed initially in the indirect ELISA, the OD450 values were significantly higher than at 0, 50, 100, and 200 μg/ml, and the background was much lower. We set out to optimize the time and temperature required for the IPK pretreatment that can be used for analyzing clinical samples. LAM spiked urine was pretreated with IPK at 400 μg/mL for a time course of 0, 10, 30, and 60 min at room temperature and 55° C. We observed that as compared to the 0 min pretreatment at room temperature and 55° C., there was no significant difference in the absorbance values at different time points and temperatures. This led us to conclude that for C-ELISA of urine clinical or spiked urine samples using IPK as pretreatment, 400 mg/mL at room temperature for 10-30 min should be optimal to achieve the release of LAM from urine. However, since the surface area of the strips is small (application volume max-5 μL), a 30 min exposure of one strip per 200 μL sample size may be more desirable.

Clinical Samples. We have shown repeatedly that during assay or method development, clinical samples do not perform in a similar manner to the control urine sample spiked with LAM. To test the newly developed procedure for sample pretreatment (IPK), 25 clinically characterized urine samples were analyzed from TB patients or suspects that had been previously validated using chemical derivatization method developed utilizing gas chromatography/mass spectrometry (GC-MS). Of these 25 urine samples, 12 were smear and culture positive and 13 were non-TB. These 25 samples (100 μL each) were pretreated simultaneously with SPK and IPK and C-ELISA performed on all. Unlike the control LAM-spike NEU, OD values for IPK were higher than for SPK. Nonetheless, in both methods, the results from 25 urine samples were in agreement with the clinical status. As expected, OD450 values for both IPK and SPK were lowest (between 0.1 and 0.2) for the LAM-negative samples. These values clustered together in a scatterplot of IPK vs SPK and were distinctly separate from OD450 values for the LAM-positive samples. Because the low values group together separately from the higher values, correlation is high: ρ=0.80, p=<0.0001, τ=0.59, p=<0.0001). However, it is useful to examine each of the LAM-positive and LAM-negative groups separately. Within the cluster of LAM-negative samples, the OD values are not correlated (ρ=−0.10, p=0.74; τ=−0.10, p =0.68). For the LAM-positive samples, the OD values were widely spread out, and moderately correlated (ρ=0.57, p=0.05; τ=0.44, p=0.05). In particular, sample 7 had a much higher OD450 value using IPK and was considered as an outlier. The sample set test was done as a initial screen for method feasibility and not a true validation. With IPK, time taken for the assay was reduced from approximately an hour to 30 min. The strips were an improvement over SPK as unbound enzyme was not found in the wash solutions that would affect the Abs in use and strips can be incorporated into lateral flow devices. The ease of use of IPK cannot be understated.

### Materials and Methods

Clinical sample cohort/Ethical statement. Anonymized archived urine samples used in this study were provided in 2014 by the Foundation for Innovative New Diagnostic (FIND, Geneva) and stored in Colorado State University (CSU). The study samples were collected from patients with symptoms of pulmonary tuberculosis presenting prior to the initiation of treatment at clinics in Vietnam, South Africa and Peru. All human urine specimens were collected from adult participants of both sexes suspected of pulmonary TB, with and without HIV co-infection. Urine specimens after collection were sedimented by centrifugation and the supernatant was stored at −80° C. within a few hours of collection. Final diagnosis (TB vs. non-TB) was established on the basis of microscopy plus >2 sputum cultures and clinical and radiologic examinations. TB was defined as being culture positive from at least one sample. Non-TB was defined as being smear and culture negative on all samples and having improved clinically/radiologically without TB-specific therapy. Patients without a firm final diagnosis (e.g., contaminated culture, persistent symptoms despite repeated negative TB cultures, or treatment for TB without culture-confirmation) were excluded from study.

Additional urine control samples were obtained from healthy volunteers from a TB non-endemic region (NEU), aliquoted and stored frozen at −80° C. until further use. The control urine was spiked with Mtb CDC1551 LAM (ranging from 0.001 μg/mL-1 μg/mL for indirect ELISA and (0.02 ng/mL to 12.5 ng/mL for C-ELISA) for optimization of the IPK pretreatment conditions and to generate an assay standard curve by serially diluting the LAM two-fold to obtain a concentration range in comparison to the unspiked urine which was used as a background negative control.

Proteinase K immobilized on a porous substrate (IPK). Proteinase K was immobilized on Whatman paper no. 1 (or on other component of the microfluidic device e.g., double-sided adhesive film and/or the hydrophilic polyester film) via a covalent bond as described as described, for example, in Küchler et al., ACS Appl Mater Interfaces. 2015; 7(46):25970-80. Whatman paper no. 1 was cut into 3×5 mm strips and 5 μl of 2.10 M lithium chloride in 0.04 M sodium periodate was dropped on the strips to modify the functionality of paper from hydroxyl to aldehyde group and maintained in dark for 30 min. After 30 min of oxidation, treated paper was washed with sterile milliQ water (MQW)×3 followed by dabbing the excess water on a blotting paper. ProK (dry powder) in 2 μL was immobilized at the required concentration/s (0-1000 μg/mL) onto the modified paper and incubated in dark for 30 min followed by washing with MQW. Sodium cyanoborohydride at 1 mg/mL (5 μL) was added to the paper strips for 5 min to obtain stable covalent bonds (secondary amines) and washed. Subsequently, the immobilized paper strips were blocked by adding 3% BSA for 15 min and washed. The immobilized paper was then dried at 37° C. and stored in a desiccator at 4° C. until further use.

LAM for assay standardization. The LAM used in this study was isolated and purified from Mycobacterium tuberculosis (Mtb) CDC1551 strain in in vitro culture. LAM was isolated in large quantities so that the same standard could be used throughout the year for recurring experiments.

Antibodies. A mouse monoclonal antibody CS35 IgG3, raised against Mycobacterium leprae whole cells, was purified from hybridoma cell line generated by the fusion of myeloma cells with immunized mouse splenocytes as described, for example, in Gaylord et al., Infect Immun. 1987; 55:2860-3.

A human mAb, A194 IgG1 was obtained from New Jersey Medical School (Rutgers University). The antibody was molecularly cloned from a patient diagnosed with pulmonary TB who had already started on drug treatment for a month before screening the culture supernatant against ManLAM in an ELISA assay using a high throughput in vitro B cell culture method.

Standard ProK pretreatment (SPK). ProK was added to the urine samples at a final concentration of 200 μg/mL and incubated at 55° C. for 30 min followed by inactivation at 100° C. for 10 min. The treated samples were then centrifuged at 12,000×g for 10 min and the supernatant used for C-ELISA. For indirect ELISA, ProK was used at 200 μg/mL to pretreat the urine spiked with LAM at 55° C. for 2 hr followed by inactivation at 100° C. for 30 min. The pretreated samples were then centrifuged at 12,000×g for 10 min and the supernatant used for the ELISA assay.

Immobilized ProK pretreatment (IPK). For optimization of IPK, ProK was immobilized on the Whatman paper #1 at varying concentrations (ranging from 0 μg/mL-1000 μg/mL) and tested in an Indirect ELISA platform using 200 μL as sample volume. A time course was setup starting from 0, 30, 60, 2, and 3 hr and pretreatment with IPK performed at room temperature. Once the optimal concentration for IPK was achieved at 400 μg/mL with optimal time between 60 to 120 min, a temperature analysis was performed for 60 mins with 400 μg/mL IPK at room temperature, 37° C. and 55° C. Best results were obtained at room temperature.

Indirect ELISA to confirm the immobilization of Proteinase K on paper strip. To optimize the concentration of ProK to be used, the optimal incubation time and temperature for the pretreatment, indirect ELISA (which measures binding of antibody to the antigens) was carried out with modifications as stated. Urine from a healthy volunteer was spiked with known amounts of LAM (ranging from 0.001 μg/mL-1 μg/mL) and pretreated with IPK strips and then used for coating a 96-well plate in equal volume of the coating buffer (0.05M carbonate bicarbonate buffer, pH 9.6) and the plate incubated at 4° C. overnight. Non-specific binding sites were blocked with 1% bovine serum albumin (BSA) in lx phosphate buffered saline (PBS) (blocking buffer) after washing the wells briefly with the same. Purified CS35 was used at a concentration of 2 μg/mL and added to all the wells and incubated for 90 min at room temperature. The plates were then washed with the wash buffer (1× PBS with 0.05% Tween-80) and then incubated for 90 min with anti-mouse IgG alkaline phosphatase conjugate for the murine primary antibody, diluted 1:2500 in wash buffer. The plates were again washed, and the alkaline phosphatase activity measured by addition of p-nitrophenyl phosphate (pNPP) as a substrate. The optical density was measured at 405nm. All standards were run in duplicates and the absorbance plotted to determine the binding activity of the antibody to the LAM. As a control for the IPK pretreatment, NEU spiked with LAM was simultaneously pretreated by addition of ProK at 200 μg/mL final concentration at 55° C. for 2 hr followed by inactivation at 100° C. for 30 min.

Capture ELISA on NEU spiked with LAM and clinical samples. For optimization of the IPK concentration, time of exposure and temperature for pretreatment on a capture ELISA (C-ELISA) platform, previously published protocol was followed with slight modifications. A 96 well polystyrene high binding plate was coated with a capture antibody (CS35 ms mAb) at 10 μg/mL concentration in PBS and incubated at 4° C. overnight. NEU spiked with known amount of LAM (ranging from 0.02 ng/mL-12.5 ng/mL) was incubated at room temperature for 30 min to allow for the complexation of LAM and urine protein/s, followed by storing at −20° C. overnight to somewhat mimic the conditions for the clinical samples. After overnight incubation, the antibody plates and the LAM samples were brought to room temperature and the plates blocked with 1% BSA in 1× PBS (blocking buffer) for 1 hr after briefly washing the plates with the same. Control and/or clinical samples were pretreated with ProK using the SPK method and simultaneously the samples were pretreated with IPK by the addition of the strip into the sample tube for the required time and the samples then used for ELISA. The plates were washed with the blocking buffer, the control and/ or the clinical samples were added to the appropriate wells and incubated for 90 min at room temperature. The plates were then washed with the wash buffer (1× PBS-0.05% Tween-80) and the biotinylated detection antibody (A194hu IgG1) added at a concentration of 250 ng/mL to all the wells and the plates incubated for 90 min at room temperature. Following another wash with the wash buffer, 1:200 dilution of Streptavidin-Horseradish Peroxidase (HRP) was added to the plates and incubated for 25 min at room temperature. After the final wash, Ultra TMB chromogenic substrate was added to all the wells and the plates incubated for at least 30 min and observed for color development. The reaction was stopped by adding 2M Sulphuric Acid to the wells and the plates read at 450 nm.

Statistical Methods. Correlation was evaluated with Spearman's ρ. P-values are based on a test of the null hypothesis that correlation is equal to zero. Analyses were conducted in the open software R version 4.0.4 (2021 Feb. 15) using base functions.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference, and in particular, U.S. Pat. Publication No.: US 20070042427, US 20150158026,and US 20180030552. No limitations inconsistent with this disclosure are to be understood therefrom. The invention has been described with reference to various specific and preferred embodiments and techniques. However, it should be understood that many variations and modifications may be made while remaining within the spirit and scope of the invention.

What is claimed is:

1. A microfluidic device comprising:
a testing zone comprising a nitrocellulose membrane comprising a proximal end, a distal end, and a center region, wherein the testing zone comprises an antibody zone disposed between the distal end and the center region of the testing zone, wherein the antibody zone comprises, in order from the center region to the distal end:
a detection zone comprising mobilizable detection antibodies conjugated to a labeling component and spot-dried to a surface of the detection zone;
a capture zone comprising one or more capture antibodies that are spot-dried and immobilized on a surface of the capture zone; and
a control zone comprising one or more anti-mobilizable detection antibodies that are spot-dried and immobilized on a surface of the control zone;
a substrate component and hydrogen peroxide separately spot-dried on a surface of the testing zone between the proximal end of the testing zone and the center region of the testing zone;
a sample inlet for receiving a sample comprising:
a first sample outlet intersecting with, and in fluid communication with, the center region of the testing zone;
a second sample outlet fluidly connected to a first flow channel, wherein the first flow channel is in fluid communication with the proximal end of the testing zone; and
an absorbent pad in fluid communication with the distal end of the testing zone; wherein the first flow channel has a greater length than the length of the first sample outlet.

2. The device of claim 1 wherein the labeling component is selected from the group consisting of a chemiluminescent agent, a particulate label, a colorimetric agent, an energy transfer agent, an enzyme, a fluorescent agent, and a radioisotope.

3. The device of claim 2 wherein the labeling component is an enzyme comprising a peroxidase enzyme or a phosphatase enzyme.

4. The device of claim 1 wherein the substrate component is a colorimetric agent.

5. The device of claim 1 wherein each of the mobilizable detection antibodies further comprise a mixture of a water-soluble polymer and a surfactant.

6. The device of claim 5 wherein the water-soluble polymer is polyvinylpyrrolidone and the surfactant is Triton X-100.

7. The device of claim 6 wherein the mixture comprises about 1% v/v to about 8% v/v of the polyvinylpyrrolidone and about 1% v/v to about 8% v/v of the Triton X-100.

8. The device of claim 1 further comprising proteinase K disposed on a surface of one or more of the sample inlet, the first sample outlet, the second sample outlet, the first flow channel, or a combination thereof.

9. The device of claim 1 wherein the nitrocellulose membrane tapers from a first width comprising the detection zone to a second width comprising the capture zone and the control zone.

10. The device of claim 1 wherein the nitrocellulose membrane is about 15 mm to about 35 mm in length.

11. The device of claim 1 wherein a second flow channel is disposed between the first sample outlet and the center region of the testing zone, wherein the length of the first flow channel is greater than a combined length of the first sample outlet and the second flow channel.

12. A method of detecting a target analyte in a test sample comprising:
a) contacting the device of claim 1 with the test sample comprising one or more target analytes and one or more buffer components, wherein the test sample is received in the sample inlet, wherein a first fraction of the test sample migrates by capillary action through the first sample outlet to contact the center region of the testing zone, wherein the first fraction of the test sample flows toward both the proximal end of the testing zone and the distal end of the testing zone, and wherein the first fraction of the testing sample rehydrates and spreads desorbed mobilizable detection antibody conjugated to a labeling component over the antibody zone;
b) binding the desorbed mobilizable detection antibody to the one or more target analytes to form an analyte-antibody complex, wherein the analyte-antibody complex then binds to the immobilized capture antibody, and the immobilized anti-detection antibody specifically binds to desorbed and unbound mobilizable detection antibody;
c) migrating, by capillary action, the second fraction of the test sample through the flow channel towards the distal end of the testing zone such that the second fraction rehydrates, spreads, and mixes the substrate component and the hydrogen peroxide over the testing zone;
d) detecting a signal from the analyte-antibody complex bound to the immobilized capture antibody, the desorbed and unbound mobilizable detection antibody bound attached to the immobilized anti-detection antibody, or a combination thereof, wherein a detectable signal from both the analyte-antibody complex bound to the immobilized capture antibody and the desorbed and unbound mobilizable detection antibody attached to the immobilized anti-detection antibody indicates the presence of the target analyte in the test sample.

13. The method of claim 12 wherein the test sample is about 75 μl to about 95 μl in volume.

14. The method of claim 12 wherein the target analytes comprise one or more of a protein, a peptide, an amino acid, a nucleic acid, a carbohydrate, a hormone, a steroid, a vitamin, a drug, a pollutant, or a pesticide.

15. The method of claim 12 wherein the target analytes comprise one or more of a protein, a peptide, an amino acids, a nucleic acid, a carbohydrate, or an organic compound derived from a bacterial pathogen, viral pathogen, or fungal pathogen.

16. The method of claim 12 wherein the test sample is a urine sample and the target analyte comprises lipoarabinomannan from Mycobacterium tuberculosis.

17. A method of determining the presence or absence of a target analyte in a test sample comprising contacting the device of claim 1 with a sample;
forming a complex comprising the target analyte specifically bound to the mobilizable detection antibody; and
measuring a detectable signal produced by:
a) both the complex specifically bound to the immobilized capture antibody and the mobilizable detection antibody not attached to the complex that specifically binds to the immobilized anti-detection antibody; or
b) the mobilizable detection antibody not attached to the complex that specifically binds to the immobilized anti-detection antibody;
thereby determining the presence of the target analyte in the test sample if the detectable signal is produced as recited in part a) and the absence of the target analyte in the test sample if the detectable signal is produced as recited in part b).
